(19) United States
(12) Patent Application Publication
(10) Pub. No.: US 2006/0008865 A1
(43) Pub. Date: Jan. 12, 2006

(54) Screening apparatus for water treatment with membranes

(76) Inventors: Pierre Lucien Cote, Dundas (CA); Doug Joseph Thompson, Hamilton (CA); Jennifer Lynn Pawloski, Guelph (CA); Minggang Liu, Burlington (CA)

Correspondence Address: BERESKIN AND PARR, 40 KING STREET WEST, BOX 401, TORONTO, ON, M5H 3Y2, CA

(21) Appl. No.: 11/168,405

(22) Filed: Jun. 29, 2005

Related U.S. Application Data
(60) Provisional application No. 60/584,168, filed on Jul. 1, 2004.
(60) Provisional application No. 60/585,579, filed on Jul. 7, 2004.
(60) Provisional application No. 60/585,580, filed on Jul. 7, 2004.
(60) Provisional application No. 60/612,515, filed on Sep. 24, 2004.
(60) Provisional application No. 60/618,980, filed on Oct. 18, 2004.

(30) Foreign Application Priority Data
Feb. 17, 2005 (CA) 2,497,391

(51) Int. Cl. C12Q 1/04; B01D 35/00

(52) U.S. Cl. 435/34; 210/295

(57) ABSTRACT

A static screen used upstream of a membrane assembly within a water treatment system has a screening surface with a number of openings distributed over its area. Liquid flows through the screening surface to reach the membrane assembly. Various shapes of screening surfaces are described including undulating panels and geometric shapes. Methods for cleaning the screen are described including aeration and backwashing. Various treatment systems or process designs incorporating the screen are described.

This is an application claiming the benefit under 35 USC 119(e) of U.S. Application Ser. Nos. 60/584,168, filed Jul. 1, 2004; 60/585,579, filed Jul. 7, 2004; 60/585,580, filed Jul. 7, 2004; 60/612,515, filed Sep. 24, 2004; and, 60/618,980, filed Oct. 18, 2004. U.S. Application Ser. Nos. 60/584,168; 60/585,579; 60/585,580; 60/612,515; and, 60/618,980 are incorporated herein, in their entirety, by this reference to them. Canadian Application Serial No. 2,497,391 filed Feb. 17, 2005 by Zenon Environmental Inc. is incorporated herein, in its entirety by this reference to it.

### FIELD OF THE INVENTION

This invention relates to screens, to a process of operating or cleaning a screen and to a water treatment apparatus or process using screens, for example a water treatment apparatus or process using membranes.

### BACKGROUND OF THE INVENTION

The following description of the background of the invention is not an admission that anything discussed in the description is citable as prior art or part of the knowledge of persons skilled in the art in any country.

Some water treatment systems include a number of membrane assemblies that may contain a number of membrane fibers or sheets. The membrane fibers or sheets are held in place, typically through headers or frames, within a larger assembly which may be called an element, module or cassette. The membrane fibers or sheets can be damaged by trash, roped hair and other fibrous materials that may become entangled with or around the membrane fiber or sheet. Moreover, trash, hair and other fibrous materials are difficult to remove from membranes because the membrane fibers or sheets are arranged relatively close to one another and cannot withstand repeated vigorous mechanical cleaning.

Reducing the build-up and entanglement of trash, hair and other fibrous materials within membrane assemblies is desirable for efficient operation and longevity of a water treatment system.

One process for reducing the build-up of hair, trash and other fibrous materials includes pre-screening a raw feed stream before it enters a membrane bioreactor. However, pre-screening the feed stream is typically only effective in reducing the concentrations of trash and other fibrous materials that are roped or balled together in the feed. Pre-screening the raw sewage stream does not adequately remove individual strands or small bundles of trash and fibrous materials that can later come together to form relatively thick roped lengths or balled bundles inside the waste water treatment system. That is, a pre-screening filter permits individual strands of hair, for example, to easily pass into a water treatment system. Once inside the water treatment system the individual hairs are prone to roping and balling together. The roped hairs become entangled with the membrane fibers causing wear and damage. Pre-screening the raw sewage stream typically requires that the screen be designed to accommodate peak raw sewage flow rates that are typically many times higher than the average flow rate Q through the waste water treatment system but the screen operates at most times under much lower flows. Additionally, recontamination of the pre-screened water is common since the water may pass through open tanks included in many water treatment facilities. Debris such as leaves from nearby trees or other contaminates brought by the wind frequently blows into the tanks. Further, the mechanical design of screens themselves may make them expensive or difficult to install or operate, particularly at high flows and fine mesh sizes.

U.S. Pat. No. 6,814,868 describes a process for reducing a trash or fibrous materials concentration in a wastewater treatment system having a membrane filter in conjunction with a bioreactor. The process comprises flowing a portion of mixed liquor through a screen in a side stream. The flow rate of the mixed liquor through the screen is about no more than the average design flow rate of the wastewater treatment system. The screenings can be either treated or disposed of directly or in combination with the waste activated sludge. The openings of the screen are between about 0.10 mm and about 1.0 mm in size as can be provided by, for example, a rotary drum screen.

### SUMMARY OF THE INVENTION

The following summary is intended to introduce the reader to the invention but not to define it. The invention may reside in a combination or sub-combination of one or more apparatus elements or process steps found in any part of this document. It is an object of the invention to improve on, or at least provide a useful alternative to, the prior art. It is another or alternate object of the invention to provide a screening apparatus. It is another, or alternate, object of the invention to provide a process for operating or cleaning a screen. It is another, or alternate, object of the invention to provide a water treatment apparatus, system or process using a screen, for example a water treatment apparatus, system or process having a membrane assembly.

According to an, aspect of the invention, there is provided a screening apparatus for use in a water treatment system having an upstream area under ambient pressure with a first static head and a downstream area under ambient pressure with a second static head, the screening apparatus comprising:
one or more generally static screening surfaces having a plurality of openings, wherein any dimension of the openings is approximately 3 mm or less;
a structure for holding the screening surface in communication with the upstream and downstream areas such that the screening surface intercepts water flowing between the upstream and downstream areas; and, one or more aerators in communication with the upstream area.

According to another aspect of the invention, there is provided an apparatus comprising:
one or more fluidly connected tanks;
an inlet to the one or more tanks;
a membrane assembly immersed in one of the tanks;
a static screen separating a volume of water containing the membrane assembly from the inlet;
a permeate outlet connected to the membrane assembly; and, a membrane retentate outlet in communication with the volume of water containing the membrane assembly.

According to another aspect of the invention, there is provided a process for treating water comprising the steps of:
flowing water containing undesirable solids, the undesirable solids being at least 20 μm wide in any direction, through a generally stationary screening surface in a forward direction from an upstream side of the screening surface to a downstream side of the screening surface, the flow of the water driven substantially by the difference between a static head in communication with the upstream side of the screening surface and a lesser static head in communication with the downstream side of the screening surface; and,
stopping the flow of water through the screening surface in the forward direction from time to time and removing undesirable solids from the upstream side of the screening surface while the flow of water through the screening surface in the forward direction is stopped.

The smallest dimension of the openings is approximately 3 mm or less, approximately 1 mm or less, approximately 250 microns or less, approximately 100 microns or less or approximately 50 microns or less.

The screening surface may be flat and may be made, for example of a wire or fibre mesh, or screen or perforated plate. Alternatively, the screening surface may comprise an undulating panel of material. Further alternatively the screening surface may have other shapes, such as an open ended three-dimensional figure, for example a cylinder. The screening surface may have an area that is twice the cross-sectional area of the screening apparatus or more. The screening surface may be cleaned without the use of moving mechanical parts acting directly on the screening surface. A static screen may have a screening surface and a non-porous surface, the non-porous surface extending vertically from below a downstream water level to above an upstream water level.

The screening surface may be arranged at an angle to a vertical axis, for example with an upper part of the screen angling upstream. The screening apparatus may also communicate with an upstream or downstream area through a header, manifold, plenum or conduit.

The upstream aerator may provide air scouring of the screening surface during forward operation or cause a backwash of the screening surface during a cleaning or deconcentration procedure. The screening apparatus may further have an overflow weir or drain upstream of the screening surface for removing solids retained by the screen, for example during deconcentration or cleaning procedures. Solids retained by the screen in an upstream area may be sent to a waste stream or re-cycle to other parts of the system. Some of these elements may be combined. For example, an aerator may simultaneously scour the screening surface with bubbles, float screenings in the upstream area to an overflow to assist in their removal or recycle, and cause a backwash of the screen.

Other aspects or features of the present invention may reside in any possible combination or subcombination of elements or steps from the set of all elements or steps described above or in any other part of this document, for example the claims.

### BRIEF DESCRIPTION OF THE DRAWINGS

Exemplary embodiments of the invention will be described below with reference to the following figures:

FIG. 1A is a schematic plan view diagram illustrating a waste water treatment system;

FIG. 1B is a schematic plan view of a filtration system;

FIGS. 1C, 1D and 1E are schematic plan views of alternate waste water treatment systems;

FIG. 2A is a schematic diagram illustrating a side view of a membrane tank shown in FIG. 1A;

FIG. 2B is a schematic plan view of an alternate membrane tank.

FIG. 2C is a schematic side view of a further alternate membrane tank and wastewater treatment system with a screening apparatus.

FIG. 3 is a schematic diagram illustrating various views of a flat panel static screen;

FIG. 4A is a schematic diagram illustrating various views of a undulating panel static screen; and

FIG. 4B is a schematic diagram illustrating an enlarged portion of the undulating panel static screen shown in FIG. 4A.

FIGS. 5A and 5B are schematic diagrams in elevation and isometric view of another waste water treatment system.

FIG. 6 is a schematic diagram of another wastewater treatment system.

FIGS. 7A and 7B are schematic diagrams in elevation and plan views of a primary screen-clarifier.

FIGS. 8A and 8B are schematic diagrams in elevation and plan views of another primary screen-clarifier.

FIG. 9 is a schematic side view of a screening apparatus.

FIG. 10 is a schematic representation of static screens of various configurations.

FIGS. 11 to 14 are graphs of experimental results.

### DETAILED DESCRIPTION OF EMBODIMENTS OF THE INVENTION

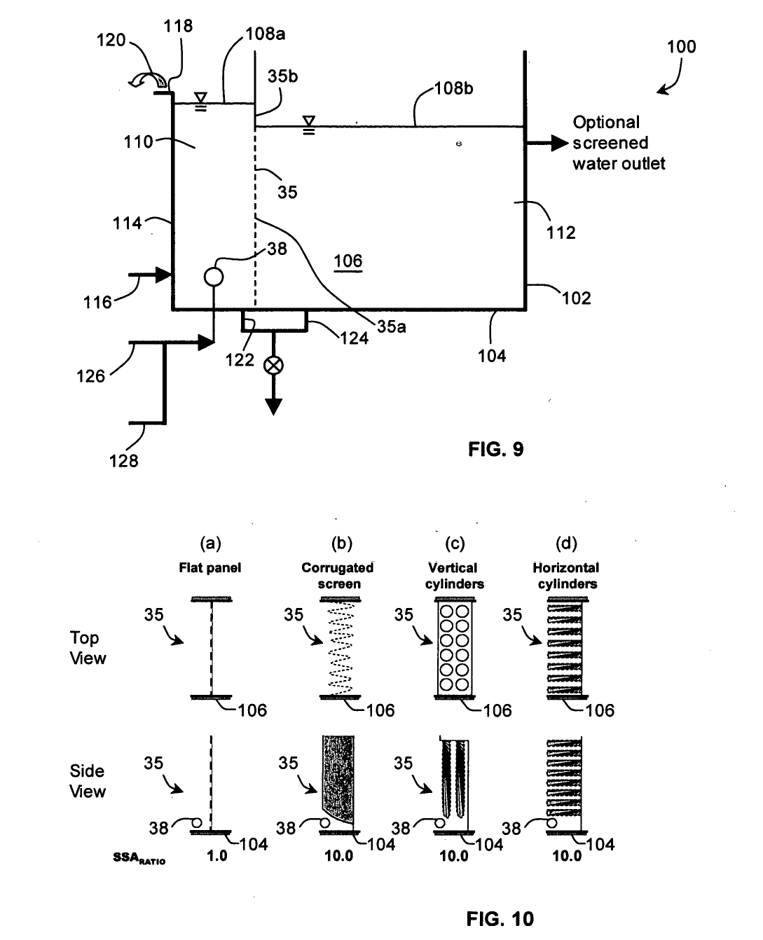

FIG. 9 shows a screening apparatus 100 having a static screen 35 mounted in a vessel 102. The vessel 102 may be, for example, a tank, trough, channel or other conduit or holding means for water. The vessel 102 has a bottom 104 and a pair of opposed sides 106, the closer of the two opposed sides 106 not shown, defining a pathway for water to flow through the vessel 102 by generally open channel flow. The sides 106 may be curved, as in a round tank. The static screen 35 spans between the opposed sides 106 either directly or by spanning between partitions or other non-porous elements attached to the sides 106. The static screen 35 also extends from the bottom 104 of the vessel to above a surface level 108 of the water in the vessel 102, either directly or by extending between non-porous elements attached to the bottom 104 or across a higher elevation of the vessel 102. In particular, the static screen 35 may have a screening surface 35a and a non-porous surface 35b. Water passing through the pathway, or from one end of the vessel 102 to another, is made to pass through the static screen 35, particularly the screening surface 35a. In this way, the static screen 35 separates the vessel 102 into an upstream section 110 and a downstream section 112. Either the upstream section 110, the downstream section 112 or both may be shared with other elements of a water treatment system. For example, the downstream section 112 may function as a membrane tank.

The non-porous surface 35b may extend from below the downstream water level 108b to above the upstream water level 108a. The non-porous surface 35b may cover between about 5% to 25% of the height of the static screen 35. The non-porous surface 35b serves to prevent water in the upstream section 110 above the water level 108b in the downstream section 112 from flowing to the downstream section 112. This assists in creating an airlift in the upstream section 110 when the upstream section 110 is aerated and is believed to improve the effectiveness of the backwash, particularly in upper parts of the static screen 35. In the absence of a distinct non-porous surface 35b, trash or other solids etc. may accumulate on an upper section of the screening surface 35a and eventually act as a non-porous section 35b. It is not necessary to use moving mechanical parts in contact with the screening surface 35a to clean the static screen 35.

During forward operation, a difference in static head between the water level 108a in the upstream section 110 and the water level 108b in the downstream section 112 drives the flow of water through the static screen 35. This head difference may be low, for example 30 cm or less, or between 15 and 30 cm. The water level 108 may be generally in the range of 2 to 4 metres.

The screening apparatus 100 may have an upstream barrier 114 which may be a partition or, as shown, an end wall of the vessel 102. The barrier 114 and the most downstream surface of the screen 35 may be located near each other, for example between 15 cm and 2 m apart, such that the upstream section 110 may have a relatively small volume compared to the downstream section 112. For example, the upstream section 110 may have a volume that is 30% or less than the volume of the downstream section 112. Particularly where the downstream section 112 contains membrane assemblies, the upstream section 110 may have a volume between about 2% to 20%, for example about 10%, of the volume of the downstream section 112. The specific size of upstream and downstream sections 110, 112, or their relative volumes, may be designed by noting that if all flow to the membrane assemblies pass through the static screen 35, then the flow to the membranes (in m³/d) is equal to (a) the product of screen specific surface area (m² screening surface 35a per m³ upstream section 110 volume), the screen flux (m/d) and the volume of the upstream section (m³) which is in turn equal to (b) the membrane specific surface area (m² membrane surface area per m³ volume of the downstream section 112) the membrane flux (m/d) and the volume of the downstream section 112. Membrane specific surface areas and fluxes may range from, for example, about 50-400 m²/m³ and 0.5-2.0 m/d respectively. Screen specific surface area may range from, for example, about 3-30 m²/m³, or be typically about 10 m²/m³, and screen flux may range from about 50-200 m/d, with a typical value about 100 m/d. Alternately, or additionally, the dimensions of the upstream and downstream sections 110, 112 may be designed noting that between about 15 and 150%, for example 20-70%, of the volume of the upstream section 110 may flow through the static screen 35 from the downstream section 112 during a backwash, to be described below. This flow should not decrease the water level 108b in the downstream section 112 excessively, for example by not more than about 20 cm or 10 cm or 7% of the ordinary water level 108b of the downstream section 112.

An inlet 116, which may be, for example, a pipe or hole or space below a partition, allows influent water or feed to enter the upstream section 110, for example from near the bottom of the upstream section 110. An overflow 118, which may be a low wall, weir, pipe, channel, or other feature, may allow water containing retained screenings, which may form a waste, reject or recycle stream 120, to leave the upstream section 110 other than by passing through the static screen 35 when the water level 108a in the upstream section 110 rises to above the bottom of the overflow 118. Primary 122 and secondary 124 drains may allow the upstream section 110 and downstream section 112, respectively, to be drained. The drains 122, 124 may be valved collectively, as shown, or individually to allow the drains 122, 124 to be opened separately. An aerator 38, for example a coarse bubble aerator, may be located in the upstream section 110, for example near the bottom 104 of the vessel 102 and near the static screen 35. The aerator 38 may be fed at different times by a filtration gas flow 126 or a backwash gas flow 128 or both. The gas flows 126, 128 may come from a single source, for example a variable speed blower, multiple independently controlled blowers, or flow control valves connected to a source of pressurized air. The filtration gas flow 126 may be in the range of between no flow and one half of the rate of the backwash gas flow 128.

The screening apparatus 100 may operate in repeated cycles of screening and backwashing. The screening may be dead end screening, that is with a volume of water generally equal to the volume of water entering the upstream section 110 passing through the static screen 35 during a filtration period. Alternately, there may be a flow of reject 120 during some or all of a filtration period, either over the overflow 118, through the primary drain 122 or through another outlet, but with water continuing to flow to the downstream section 112 through the static screen 35. The filtration gas flow 126 may be provided continuously or intermittently at a low level during filtration to decrease the rate of reject build up on the static screen 35 while still permitting water to flow forward, that is towards the downstream section 112, through the static screen 35. As rejected materials build up on the static screen 35, the head difference between the water levels 108a, 108b will increase if a constant flow through the static screen 35 is maintained, or flow through the static screen 35 will decrease. In either case, performance may be fully or partially restored by backwashing the static screen 35. Backwashing can be, for example, at fixed intervals, for example as controlled by a timer, or triggered by reaching a preset water level 108a in the upstream section 110, or a decline in flow or another parameter.

The required backwash frequency is related to screen loading rates, trash tolerance, screen surface area and upstream section 110 volume. For example, a pilot system had a screen surface area of 5.4 ft² operating at a screen loading rate of 5.5 gpm/ft² which allowed for a trash tolerance of 3 g/L. The volume of the upstream section 110 was 75 L. The feed flow was 30 gpm (5.5 gpm/ft2×5.4 ft²) and the maximum allowed trash accumulation in the upstream area 110 was 225 g (3 g/L×75 L). With dead end screening, and a trash concentration of 150 mg/L in the feed 116, and assuming complete rejection of trash by the static screen 35, the maximum trash loading is reached in about 13 minutes, requiring backwashing every 13 minutes. Backwashing frequency may vary between 2 and 60 minutes or between 5 and 30 minutes.

Backwashing may be performed, for example, by applying the backwashing gas flow 128 to the aerator 38. The backwashing gas flow 128 may reduce the density in the water in the upstream section 110, floats solids, creates an air lift or performs a combination of two or more of these effects. For example, applying air at a rate of between 2 and 10 scfm into a 67.5 L upstream section 110 produced air to liquid rates of 3 to 20% in the water in the upstream section 110 and approximately corresponding reductions in the density of the fluid on the upstream section. The air to liquid ratio varied generally linearly with air flow rate. The backwashing gas flow 128 causes a flow reversal through the screen 35. During the flow reversal, water is removed from the upstream section 110, for example through primary drain 122 or by increase of the water level 108a in the upstream section 110 above the overflow 118, or further increase of upstream water level 108a above the overflow 118 if the water level 108a was previously above the overflow 118, to remove accumulated solids entrained in the backwash flow. At the end of a period of forward screening, the driving head may have increased to 10 to 30 cm of water column. The backwashing gas flow 128 rate may be such that the air hold-up, or the amount of air trapped in the liquid column, reduces the density of the mixture such that the static head in the upstream section 110 is below that of the downstream section 112. The backwashing gas flow 128 may be in the range of 10-50 scfm/ft² of footprint, or plan view area, of the upstream section 110. Backwash periods may last between 5 and 60 or 10 and 20 seconds. During a backwash, water entering the inlet 116 may continue to flow to, but by-pass, the static screen 35 and assist in recovering retained or rejected solids from the upstream section 110. Alternately, feed flow through the inlet 116 may be stopped during a backwash. For example, feed flow through the inlet 116 during a backwash may be between 0% and 100% or between 10% and 100% of the volume of the upstream section 110. Thus, considering feed flow and backwash flow from the downstream section 112, between 25% and 250% or between 40% to 150% of the volume of the upstream section 110 may be discharged during a backwash.

Rates of gas flows 126, 128 and allowable head through the static screen 35 are related so as to allow both forward filtration and backwashing. For example, maximum head differential, overflow 118 elevation, downstream water level 108b, and backwash gas flow 128 are related in that backwash gas flow 128, in combination with other conditions, must be sufficient to cause a backwash, with water in upstream section 110 at the overflow 118 if aeration and an overflow 118 are the method of water removal during backwash. In contrast, filtration gas flow 126 is made high enough to scour the static screen 35 and prevent quick plugging, but not so high as to reduce the effective head unnecessarily or excessively given a desired range of head differential between upstream and downstream areas 110, 112 during forward screening, overflow elevation 118 or downstream water level 108b constraints.

If the vessel 102 contains membrane assemblies in the downstream section 112, relaxing the membrane assemblies, that is reducing the rate of permeation, or stopping permeation, may be done to reduce the reduction in downstream water level 108b caused by permeation during a screen backwash. Further, backwashing the membrane assemblies may be done during a screen backwash to add water to the downstream section 112 and may temporarily raise the water level 108b in the downstream section 122. In some systems, and optionally with feed 116 to the upstream section 110 temporarily stopped, backwashing the membrane assemblies can cause a backwash of the static screen 35 alone or assist in keeping the water level 108b in the downstream section 112 high during a backwash. To use this effect, a controller controlling the screen backwash process, for example by controlling when the backwash gas flow 128, may communicate with a controller controlling the membrane permeation or backwash processes such that screen backwashing and membrane relaxation or backwashing occur wholly or partially sequentially, simultaneously or generally near each other in time, for example with the membrane backwash or relaxation starting slightly before or with the screen 35 backwash. In this case, the screen 35 backwash frequency may match a fraction or multiple of a membrane backwash or relaxation frequency. Parameters, such as screen opening size, screen loading rate, upstream section recirculation flow, screen aeration rate during filtration, fixed solids loading, etc. may be adjusted to make an even fraction or multiple of the membrane backwash or relaxation frequency acceptable as the screen backwash frequency.

The screening apparatus 100 is useful, among other things, for combination with a membrane water treatment system. The screening apparatus 100 protects downstream membranes. The screening apparatus 100 may be placed directly in front of the membranes to protect them from contamination in upstream parts of the treatment system, for example by placing membrane assemblies in the downstream section 112. In addition to protecting the membranes, the screening apparatus 100 may allow the membranes to be packed at a higher density or operated at increased flux or reduced cleaning or aeration. The screening assembly 100 may replace, remove or reduce the need for head works screening. The static screen 35 may have openings of 3 mm or less. Round or square openings are preferred although other shapes may also be used. Opening size of punched holes is taken as the diameter of round holes or the smallest width of the opening of holes that are not circular. Opening size of an opening in a mesh is taken as the width between edges of the mesh fibers if using a square mesh, or across the shortest width if the openings are rectangular. Non-round punched holes or rectangular mesh openings preferably do not have a width of opening in any direction more than 5 times, or more than 2 times, the smallest width of opening.

For the purposes of this document, the word “trash” refers to solid particles of 1 mm or more in any dimension. However, a screening apparatus 100 may also protect membranes from other undesirable solids. The words “undesirable solids” refer in this document to any solid having any dimension of 20 μm or more. Trash and undesirable solids may be originally present in the feed water, be introduced into a water treatment system after its inlet or form in the water treatment system by combination of smaller particles. Trash may include roped or balled hair, bits of plastic, vegetation debris, or other solids. Undesirable solids may include sand particles, eggs, or other solids. In general, trash tends to be more damaging to membranes than other undesirable solids. An opening size of 3 mm or less may offer significant protection against trash. Further, the inventors have observed that solids smaller than the opening size may still be caught by a static screen. However, a smaller opening size may help operation with backwash and air scouring as the only cleaning operations. For example, openings of 1 mm or less may avoid stapling with feeds containing hair or short fibres and so reduce cleaning and maintenance needs of the static screens 35. But, much smaller openings may be difficult to clean and provide unnecessary removal of solids. For example, in the context of a membrane bioreactor where mixed liquor is screened, an opening size of 1 mm or less removes significant amounts of hair, even though the hair has a diameter of much less than 1 mm. However, an opening size of 0.5 mm or less will also remove significant amounts of paper fibers although paper fibers appear to readily pass through larger openings. The paper fibers are much less damaging than hair and may also biodegrade in the system. There may be an insufficient protection advantage to justify the increased screen head loss and maintenance of a screen surface 35b with openings of 0.5 mm or less caused by retention of paper fibers. For these reasons, the inventors prefer opening sizes of between 0.5 and 1 mm for screening mixed liquor. However, when screening surface water, for example, the solids loading is lower and biodegradation of undesirable solids does not occur and so smaller opening sizes may be used. For example, opening sizes of 250 μm or less or 100 μm or less provide enhanced protection with acceptable screen head loss and maintenance. Even smaller openings, for example 50 μm or less, or between 20 μm and 50 μm, may advantageously also remove algae or other such items and so offer increased membrane or system performance sufficient to justify further increases in screen head loss and maintenance.

The backwash or reject stream water is a diluted suspension of rejected materials and may be sent to an upstream process tank or a side stream or branch process, for example a backwash water collection tank, a clarifier, a hydrocyclone, or directly to waste. The downstream section 112 is preferably of sufficient volume such that the backwashing lowers the water level 108b on the downstream sections by only a fraction, for example ½ or less, of the maximum head differential through the static screen 35, for example by about 15 cm or less or about 10 cm or less. The backwash gas flow 128 requires a fairly large flow for a short period of time and may be provided by diverting air from an existing source or a source with other uses, for example membrane scouring air or aerobic tank air.

The attributes of the screening apparatus 100 make it ideal for the protection of membranes by continuously screening mixed liquor which will be the primary application described below. However, the screening assembly 100 may also be used for other applications. Such other applications include screening raw sewage, particularly in shipboard applications where there is a low loading rate and tankage to store feed and filtered water, or other small waste water treatment systems. The screening apparatus 100 may also be used to protect membranes filtering surface or other water to create potable or process water or performing tertiary filtration. In this case, smaller openings in the static screen 35, for example 250 microns or less or 100 microns or less, may be used to remove undesirable particles such as sand, Barnacle eggs etc. The screening assembly 100 can also be used to remove algae or floc in surface water or enhanced coagulation filtration applications. In these cases, openings in the static screen 35 may be 50 microns or less and the screening assembly 100 may provide an active separation step.

The static screen 35 may be made in a variety of shapes or configurations, for example as shown in FIG. 10. Design (a) is a simple flat screen laid across a section of a vessel 102 with proper reinforcement. Designs (b), (c) and (d) aim at increasing the screening surface area for a given cross-sectional area of the static screen 35 or a tank that the static screen 35 fits into, as defined by a “Specific Surface Area” parameter:

$${SSA}_{ratio} = \frac{Screening surface area}{Tank cross \text{-} sectional area}$$

SSAᵣₐₜᵢₒ may be about 1 in situations where a simple screen is sufficient. In more demanding applications, static screens with SSAᵣₐₜᵢₒ of 2 or more, 5 or more or 10 or more, for example between 2 and 15, may be used. Sample designs and screen areas for each of the four designs of FIG. 10 are presented in Table 1. It was assumed for this Table that the screens would be located across the front of a standard tank specified for ZeeWeed™ 500d modules by their manufacturer Zenon Environmental Inc. these tanks have a width of 3 m (10 ft) and operate at a water depth of about 2.75 m (9 ft). To simplify comparison, the 3 non-flat screens have been designed to the same SSAᵣₐₜᵢₒ of 9. Larger screening surface areas could be provided at the same SSAᵣₐₜᵢₒ by locating the static screen along the side of the tank rather than across the front of it.

TABLE 1

| | | Surface Area | |
|---|---|---|---|
| Screen Concept | Key Dimensions | m² (ft²) | SSAᵣₐₜᵢₒ |
| Flat screen (a) | Tank width: 3 m | 7.4 (80) | 0.9 |
| | Water depth: 2.75 m | | |
| | Screen fraction: 0.9 | | |
| Corrugated | Corrugation depth: 300 mm | 74 (800) | 9 |
| screen (b) | Corrugation pitch c/c: 50 mm | | |
| | # of corrugations: 60 | | |
| | Screen height: 2.3 m | | |
| | Screen fraction: 0.9 | | |
| Vertical | Cylinder diameter: 100 mm | 74 (800) | 9 |
| cylinders | Cylinder length: 2.0 m | | |
| screen (c) | # of cylinders: 117 | | |
| | c/c spacing: 125 mm | | |
| | Top plate dimensions: | | |
| | 0.6 m × 3.0 m | | |
| Horizontal | Cylinder diameter: 60 mm | 74 (800) | 9 |
| cylinders | Cylinder length: 0.5 m | | |
| screen (d) | # of cylinders: 785 | | |
| | c/c spacing: 100 mm | | |

Flat or corrugated screens may be made, for example, of wire, plastic or textile fibers, woven or welded into a mesh or fabric, or perforated plates. Cylindrical screens may also be made of, for example, wire mesh, plastic mesh or punched or molded parts. Other materials and structures may also be used.

Tests on a flat screen, as in design (a) of FIG. 10, with 0.75 mm openings, indicate that such a static screen can handle 3-6 gpm/ft², depending on cleaning frequency, trash concentration and whether there is a recirculation flow, for example of about 1 Q through the upstream section 110. Such a recirculation flow, which may flow across the face of the static screen 35 and exit through the overflow 118 or another outlet, has been found to increase acceptable loading rates by 1.5 to 2.5 gpm/ft². With a 10 ft wide tank and 9 ft water depth, and providing 3″ for structural support on all 4 sides, the flat panel static screen 35 has an area of about 80 square feet. Such a screen is suitable for applications having up to about 2 Q of flow with a tank holding a cassette 48-64 ZeeWeed™ 500 membrane elements or flows of 1 Q with two such cassettes. Suitable applications could include filtration plants, small sewage systems, or shipboard or military wastewater systems. Changing to a corrugated static screen 35 allows a higher flow or more membrane elements to be placed in the tank. For example, a corrugated static screen 35 may have a depth of 300 m, pitch of about 60 mm, height of 2.6 m, and 50 loops for a total area of about 78 m² or 845 ft². Such a screen would allow flows of 3-6 Q to be provided to tanks containing about 192 to 384 elements of ZeeWeed™ 500 membranes, or 3 to 8 cassettes, with flows through the static screen 35 of 5 gpm/ft² or less. Such a static screen 35 would be suitable, for example, for larger wastewater treatment systems.

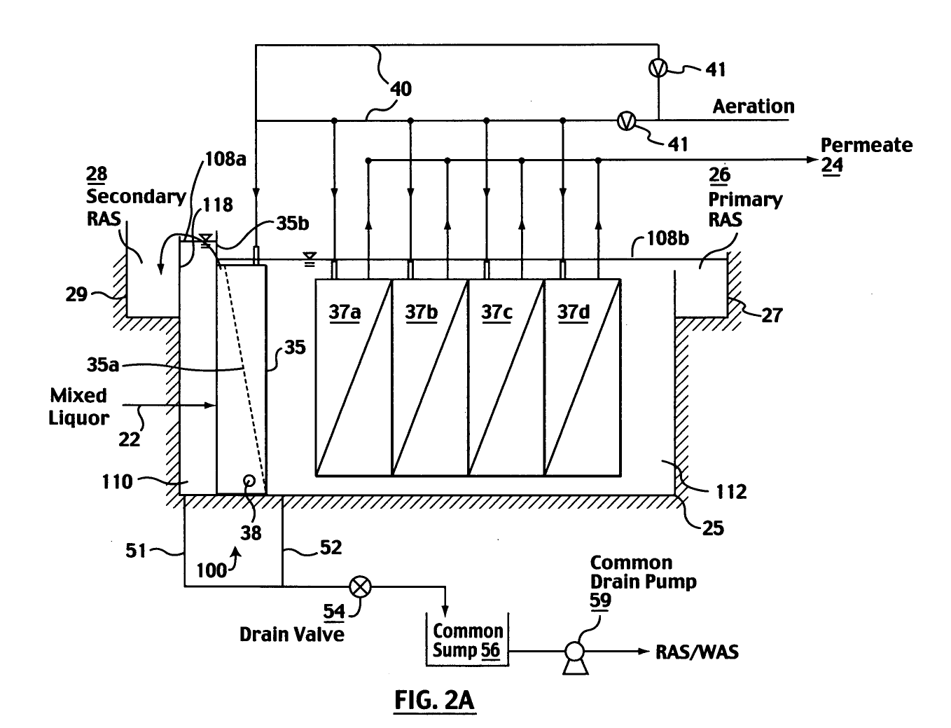

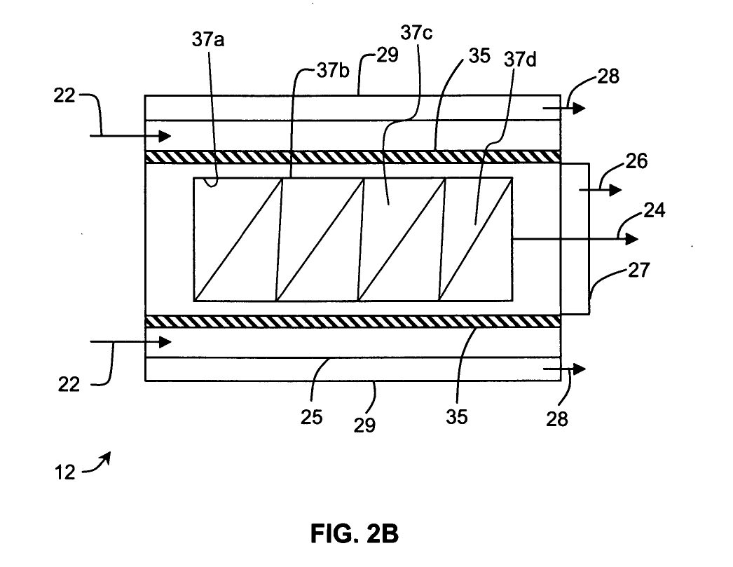

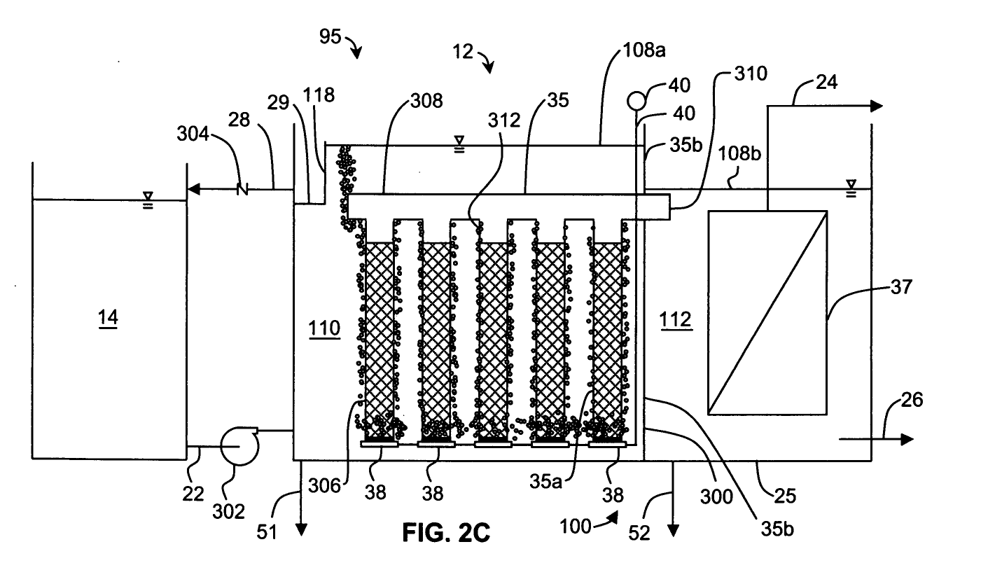

Similarly, designs according to options (c) or (d) of FIG. 10 also allow increased flow. For example, 16 cylindrical screens of 9′ height and 12″ diameter spaced at 14.5 inches centre to centre provide a screening surface area of 465 square feet. This should be sufficient to allow flows of 3-6 Q to 64 to 224 ZeeWeed™ 500 elements or 1 to 4 cassettes. In all of the cases discussed above, the number of membrane elements or flow, as a multiple of Q, can be increased by altering the plan view shape of a membrane tank. For example, if the membranes and tank walls are rearranged to make the tank larger in one dimension, the static screens 35 can be placed across the larger dimension with the inlet 116 to the tank moved to feed into the upstream area 110. For example, a static screen 35 may run down one or both edges of a long tank rather than across the front of such a tank, for example as shown in FIG. 2B. The use of one or more static screens 35 with large SSAᵣₐₜᵢₒ, locating the static screen 35 across the length of a tank, or having a cross or recirculating flow across the face of the static screen 35 may be appropriate for using the screening assembly 100 in large municipal wastewater treatment plants or other intense applications.

In operation, a repeated cycle of forward filtration and backwashing is the ordinary operation mode. During this mode of operation in a bioreactor, trash or undesirable solids of a size caught by the screening apparatus 100 build up in the biomass to a concentration generally equal to the ratio of SRT to HRT multiplied by the concentration of such solids in the feed. During an optional mode of operation, used for example at night or other periods when the flow rate is reduced, the screening apparatus 100 is run for an extended period of time, for example 1 hour or more, without backwashing. This causes the trash or undesirable solids concentration to increase in the upstream section 110. At the end of this period, the trash or undesirable solids are wasted by overflow or drain, for example to a waste activated sludge holding tank. This removes large amounts of trash or undesirable solids from the system in excess of that ordinarily removed with wasted sludge. The process may be repeated, if desired, to remove more trash. The average concentration of solids retained by the screening apparatus 100 may thus be less than the concentration described above under ordinary operation. Using this additional concentration and wasting procedure may reduce or eliminate the need for head works or side stream screening.

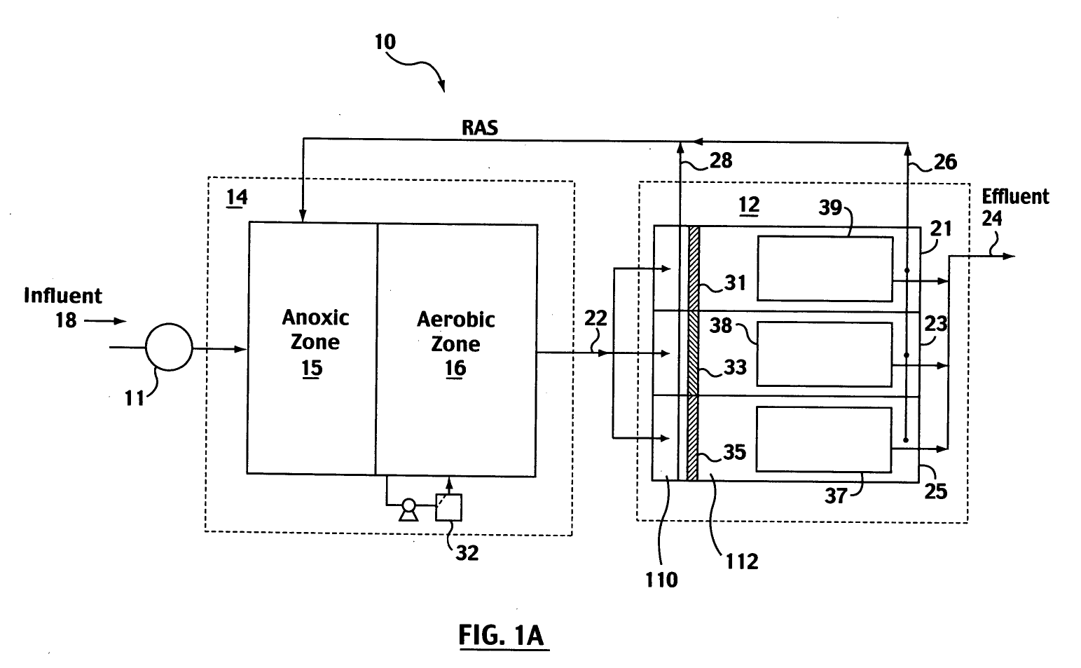

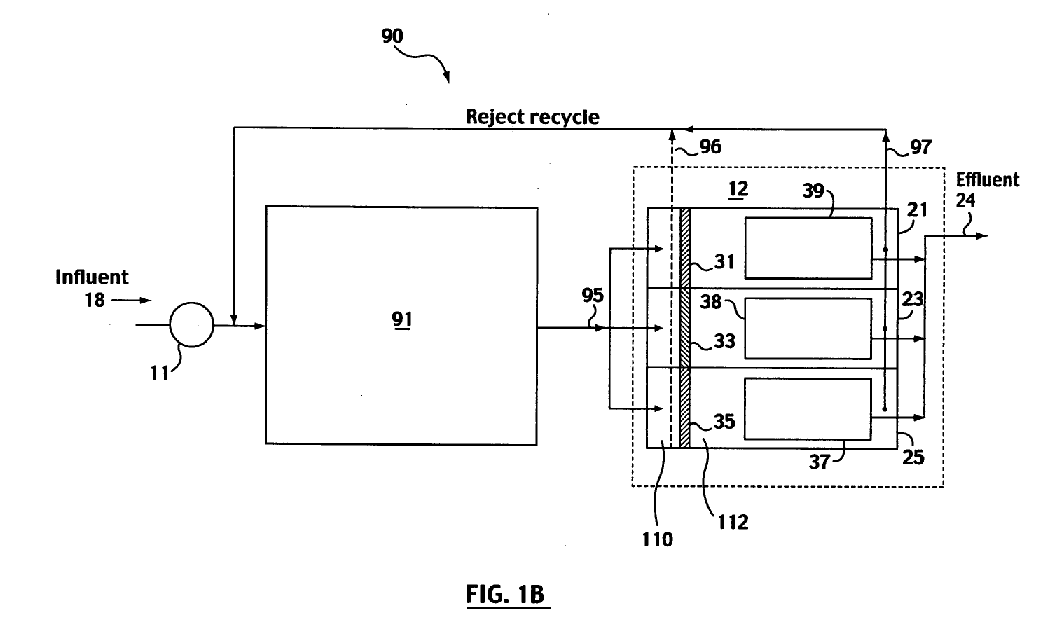

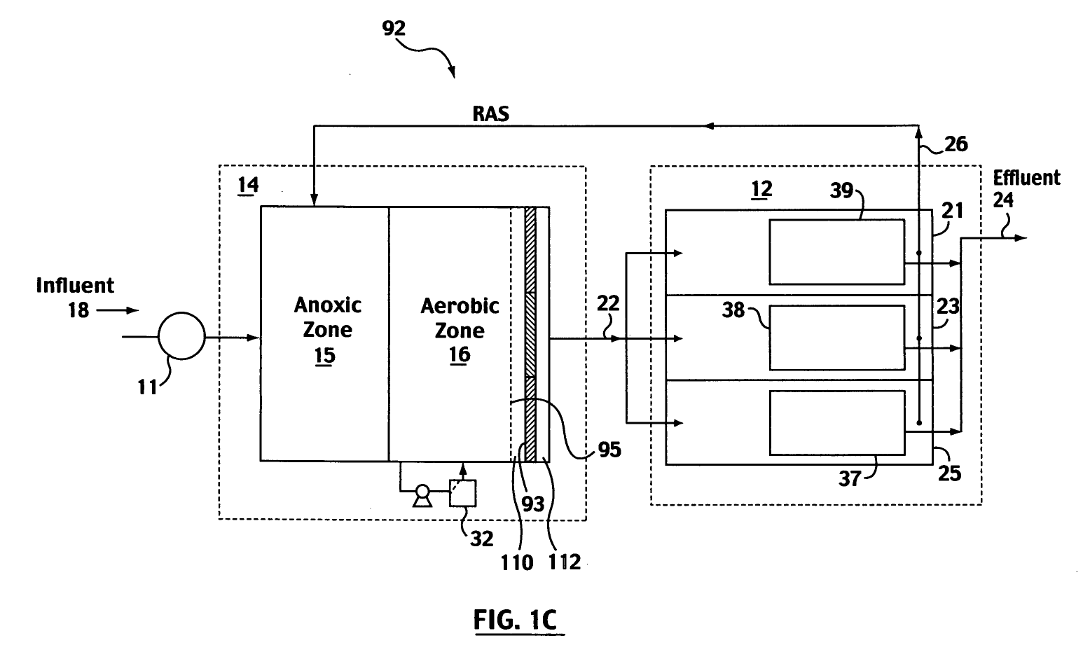

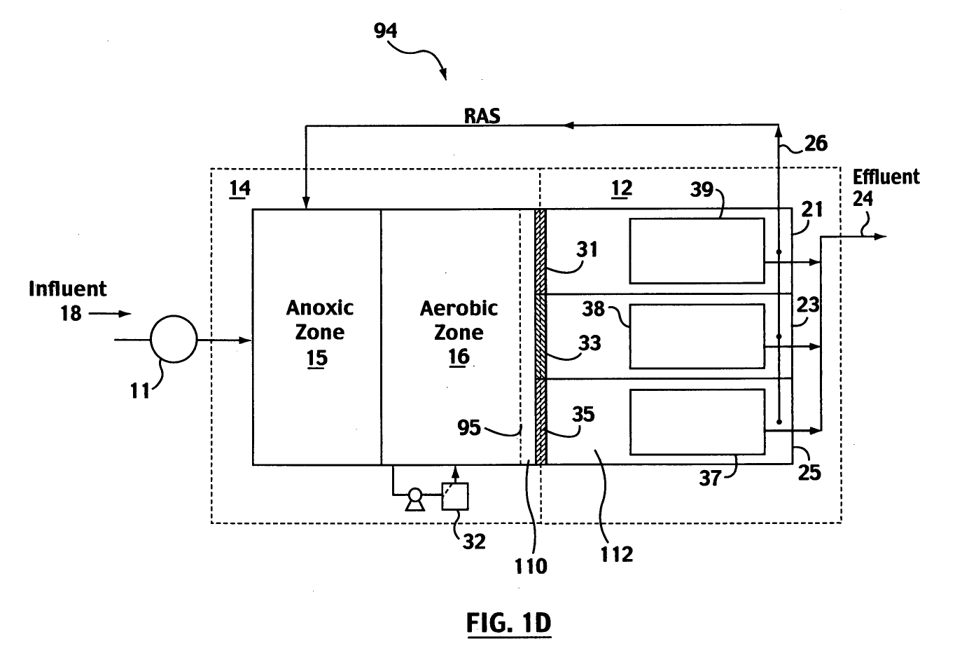

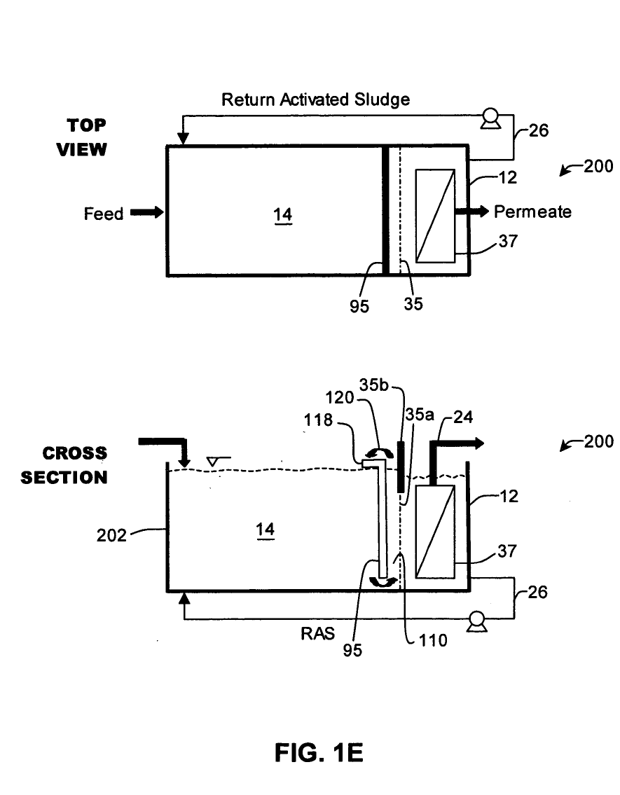

FIG. 1A is a schematic diagram illustrating an example of a waste water treatment system 10. The waste water treatment system 10 includes an optional pre-screen filter 11, a bioreactor 14 and a membrane zone 12 respectfully arranged in series but with some recycle. Briefly, raw sewage 18, alternately called influent or feed, flows into the waste water treatment system 10, optionally through the pre-screen filter 11 and treated water 24, alternately called permeate or effluent, flows out of the waste water treatment system 10 through the membrane zone 12.

In some embodiments the pre-screen filter 11 is designed to screen raw waste water 18 (i.e. raw sewage) to an input level acceptable in a conventional activated sludge plant, which typically means that debris (e.g. wood, fish, trash, hair and fiber bundles, etc.) larger than 3 mm to 6 mm in cross-section are stopped by the pre-screen filter 11, whereas smaller pieces of debris (including hair and the like) are permitted to pass through into the waste water treatment system 10. In alternative embodiments, a pre-screen filter 11 is adapted to meet the requirements for a particular facility that it is employed in. Consequently, debris smaller or larger than described above may be permitted to pass through a particular pre-screen filter 11.

Generally, the bioreactor 14 is made up of, without limitation, alone or in various combinations, one or more anaerobic zones, one or more anoxic zones, or one or more aerobic zones. According to the specific example illustrated in FIG. 1, the bioreactor 14 is made up of an upstream anoxic zone 15 that flows into a downstream aerobic zone 16. In some embodiments the sewage in one or both zones 15 and 16 is continuously stirred. The bioreactor 14 also includes an optional side-screen filtering system 32 that is provided to further reduce the concentration of hair, trash and other fibrous materials in the bioreactor 14. Details relating to a side-screen filtering system 32 are provided within the applicant's U.S. Pat. No. 6,814,868 issued on Nov. 9, 2004, which is hereby incorporated in its entirety by this reference to it.

Additionally, according to the specific example illustrated in FIG. 1A, the membrane zone 12 is fluidly connected downstream of the bioreactor 14 through exit stream 22. Flow through the exit stream 22 may be by gravity flow or pumped. The membrane zone 12 may be made up of one or more membrane tanks 21, 23 and 25 which may be separate tanks or partitioned areas of a larger tank. Membrane tanks 21, 23, 25 each have a respective static screen 31, 33 and 35. Each static screen 31, 33 and 35 sealingly covers and intercepts a respective inlet flow path for the corresponding membrane tank 21, 23 and 25 so that the amount of fibers and trash that pass into the membrane tanks 21, 23 and 25 is substantially reduced during operation. Moreover, as will be described in detail further below with reference to FIG. 2A, each membrane tank 21, 23 and 25 contains one or more respective membrane assemblies 37, 38 and 39. Each membrane tank 21, 23 and 25 is preferably designed to closely confine the respective membrane assemblies 37, 38 and 39 to reduce the required area of the membrane tanks 21, 23 and 25. For example, the membrane tanks 21, 23, 25 may have a width from 0 to 60% wider than the width of the respective membrane assemblies 37, 38 and 39.

A first number of respective outlets of the membrane assemblies 37, 38 and 39 are fluidly connected to the effluent stream 24, which is the treated water or permeate stream. A second number of respective outlets of the membrane tanks 21, 23 and 25 are fluidly connected to a common primary Return Activated Sludge (RAS) stream 26; and, similarly, a third number of respective outlets of the membrane tanks 21, 23 and 25 are fluidly connected to a common secondary RAS stream 28 or RAS by-pass. The RAS stream 26 may carry a flow of 3-5 Q. The secondary RAS stream 28 may carry flow only from backwashing the static screens 31, 33, 35, or may also carry a continuous recirculating flow of, for example, 0.5-2 Q. The primary and secondary RAS streams 26 and 28 are combined and flow back into the bioreactor 14. Specifically, in the example of FIG. 1A, the combined primary and secondary RAS streams 26 and 28 are fed back into the anoxic zone 15. In other embodiments, the feed back of RAS from any number membrane tanks may flow, without limitation, to a suitable combination of one or more anoxic zones, one or more anaerobic zones, and one or more aerobic zones or to a point upstream of the bioreactor 14.

In operation the influent stream 18 enters the waste water treatment system 10 through pre-screen filter 11 which screens the influent stream 18 so that larger pieces and bundles of debris are kept out of the waste water treatment system 10.

The screened influent stream 18 then enters the anoxic zone 15 of the bioreactor 14 where it is processed accordingly and becomes and merges with mixed liquor. Mixed liquor from the anoxic zone 15 flows to the aerobic zone 16, where it is again processed accordingly into, merges into and becomes an aerated mixed liquor.

The aerated mixed liquor exits the bioreactor 14 through exit stream 22, which is, in turn, fed into the membrane zone 12. Within the membrane zone 12 the mixed liquor is delivered into the membrane tanks 21, 23 and 25 by first passing through the corresponding static screens 31, 33 and 35, respectively. The static screens 31, 33 and 35 serve to protect the membrane assemblies 37, 38 and 39 within the respective membrane tanks 21, 23 and 25 from, for example, trash such as roped and balled bundles of hair that have formed together within the bioreactor 14 from smaller strands, smaller particles that passed through the pre-screen filter 11, or trash that has re-contaminated the bioreactor 14. As will be described in detail below with further reference to FIG. 2A, one way of dealing with the screenings that cannot pass through the static screens 31, 33 and 35 is to flush them back into the bioreactor 14 via the secondary RAS stream 28. In some embodiments, the flow rate through the secondary RAS stream 28 is about the same as the average flow rate Q, for example between 0.5 and 1.5 Q, of the waste water treatment system. However, flow in the secondary RAS stream 28 may not be at a constant rate and the flow rates in the sentence above may be averages over periods of time. For example, where the screen 25 is backwashed in a way that causes backwashed liquid or solids to flow into secondary overflow weir 29 to join the secondary RAS stream 28, as will be described further below, the flow rate in the secondary RAS stream 28 may be minimal or zero while liquid flows in a forward direction through the screen and 4-6 Q during a backwash of the screen 35. As mentioned above, a constant flow, for example of 0.5-2 Q, through the secondary RAS stream 28 may also be superimposed onto these flows. Flow in the secondary RAS stream 28 may be by gravity, for example when the membrane zone 12 is at a higher elevation than the bioreactor 14, or by pump, optionally after flowing by gravity into a well, sump or channel, for example if the bioreactor 14 is at a higher elevation than the membrane zone 12. Alternatively or additionally, screenings may be removed from the waste water treatment system 10 and disposed of as Waste Activated Sludge (WAS).

A treated effluent stream 24 exits from the permeate side of the membrane assemblies 37, 38 and 39. RAS, including material rejected by the membrane assemblies in the membrane zone 12, is fed back to the bioreactor 14 via the primary RAS stream 26. In some embodiments, the flow rate through the primary RAS stream 26 is about three or four times the average flow rate Q, for example between 2.5 Q and 4.5 Q, of the waste water treatment system. Required flow through the static screens 31, 33, 35 may be 3.5-5.5 Q. Alternatively or additionally, waste sludge may be removed from the waste water treatment system 10, for example as described further below, and disposed of accordingly.

Independently, an optional side-screen filtering system 32 may remove a portion of the mixed liquor from the bioreactor 14 in order to remove trash, hair and other fibrous materials from the mixed liquor before re-introducing the screened mixed liquor into the bioreactor 15. Specifically, as shown in FIG. 1, the side-screen filtering system 32 is coupled to remove a portion of the mixed liquor from the aerobic zone 16 of the bioreactor 14 and re-introduce the screened mixed liquor into the aerobic zone 16.

In some embodiments, a side-screen filtering system operates at a constant flow rate that may be 25% to 75% of the average flow rate Q through a waste water treatment system. In some related embodiments one or more side-screen filtering systems can be placed at various other locations within a waste water treatment system for screening the mixed liquor and subsequently re-introducing it to the same location or another location within the waste water treatment system. Again, details relating to side-screen filtering are provided within the applicant's U.S. Pat. No. 6,814,868. The side screen filtering system reduces the concentration of roped or balled hair or similar materials and other trash in the bioreactor 14, but does not eliminate them.

The flow of mixed liquor through waste water treatment system 10 can be facilitated in a number of ways. According to a first option mixed liquor is pumped from the bioreactor 14 to the membrane zone 12; and, gravity is employed to circulate the combined RAS stream back to the bioreactor 14. The level of the mixed liquor in one or more of the membrane tanks 21, 23 and 25 is controlled by the height of overflow weir 27 to the primary RAS stream 26. Advantageously, floating foam and/or scum is passively delivered back to the bioreactor 14 from the membrane zone 12 over the overflow weir 27, although other means for RAS recirculation and foam or scum control can be used. Alternatively, according to a second option, mixed liquor passively flows (e.g. assisted by gravity) from the bioreactor 14 to a membrane zone 12; and, the combined RAS stream is circulated to the bioreactor 14 using a pumping mechanism. Advantageously, in accordance with the second option, the RAS pump does not have to process the permeate flow, reducing the peak pumping requirements of the system.

Referring to FIG. 1B, a second waste water treatment system 90 has a conventional activated sludge plant (typically including a settling or clarifying step and an internal RAS line) 91 upstream of a membrane zone 12 that provides tertiary filtration of the effluent from the plant 91 through conduit 95. The membrane zone 12 of FIG. 1B is generally similar to that in FIG. 1A and like reference numerals denote the same elements as in FIG. 1A. However, the reject streams 96, 97 are returned to other parts of the plant 91. Primary reject stream 97 also carries only the membrane reject, which may be about 0.05 to 0.1 Q. Secondary reject stream 96 may be omitted or used only intermittently, for example, to return solids floated during aeration after backwashing the screens 31, 33, 35, as will be described further below. Potable, municipal or process water filtration plants may operate similarly except the activated sludge plant 91 may be omitted or replaced by pre-treatment zones such as a clarifying or settling zone or a coagulation or flocculation zone.

FIGS. 1C and 1D show further embodiments of waste water treatment systems. In FIG. 1C, treatment system 92 has a large screen 93 extending across the width of the bioreactor 14, and from the bottom of the tank to the maximum water-level, at the downstream end of the last zone (aerobic zone 16 in the embodiment of FIG. 1C), and just upstream of the outlet to exit stream 22. Secondary RAS stream 28 is omitted since retained screenings stay in the bioreactor 14. In FIG. 1D, third system 94 has a common tank for part of the bioreactor 14, the aerobic zone 16, and the membrane zone 12. The screens 31, 33, 35 act as screening partition walls between the aerobic zone 16 and the membrane tanks 21, 23, 25. Again, secondary RAS stream 28 is omitted. In both FIGS. 1C and 1D, an aerator in the screens 31, 33, 35, to be described further below, may help provide oxygen to the aerobic zone 16. An optional partition 95, to be described below in relation to FIG. 1E, may be added to aid in cleaning the screens 33, 35, 93 by backwashing.

FIG. 1E shows a small membrane bioreactor (MBR) 200. The MBR has a single composite tank containing a membrane zone 12 and a bioreactor 14 having a single aerobic zone. The bioreactor 14 is separated from the membrane zone by a static screen 35 and a partition 95. The partition creates a small volume upstream section 110 to aid in backwashing the screen 35. No secondary RAS stream 28 is provided but during backwashing, screen reject 120 flows over the top of the partition 95 which acts as an overflow 118.

Referring now to FIG. 2A, illustrated is a schematic diagram of a side view of the membrane tank 25 of FIGS. 1A, 1B and 1D that is arranged with the corresponding static screen 35 to provide an integrated screening apparatus 100. The static screen 35 is positioned close to the inlet side of the membrane tank 25 to provide an upstream section 110. Specifically, the static screen 35 extends across the width of the membrane tank 25, extending from the bottom of the membrane tank 25 to at least the design maximum mixed liquor level, and generally sealingly cooperates with the bottom and sides of the membrane tank 25. A non-porous surface 35b may extend from the top of a frame around the screen surface 35a below the downstream water level 108a to above the upstream water level 108a. In such an arrangement, the static screen 35 divides the membrane tank 25 into two portions, the upstream section 110 and downstream section 112. The upstream section 110 is fluidly connected to the exit stream 22 which is an inlet to the upstream section 110 bringing in mixed liquor, either by pumped or gravity flow. The downstream section 112 contains the membrane assemblies 37, 38 and 39 (described below). Membrane tanks 21 and 23 are substantially identical to membrane tank 25. The arrangement of the embodiment of FIG. 1C also has the features described above except that the large screen 93 extends across the entire aerobic zone 16. The membrane tank 25 is one example of how a membrane tank can be arranged in accordance with aspects of an embodiment of the invention although other arrangements may also be used.

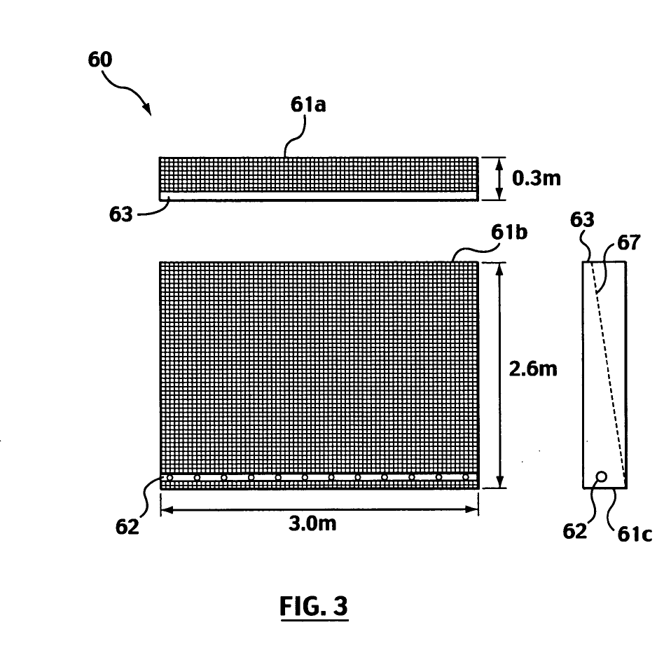

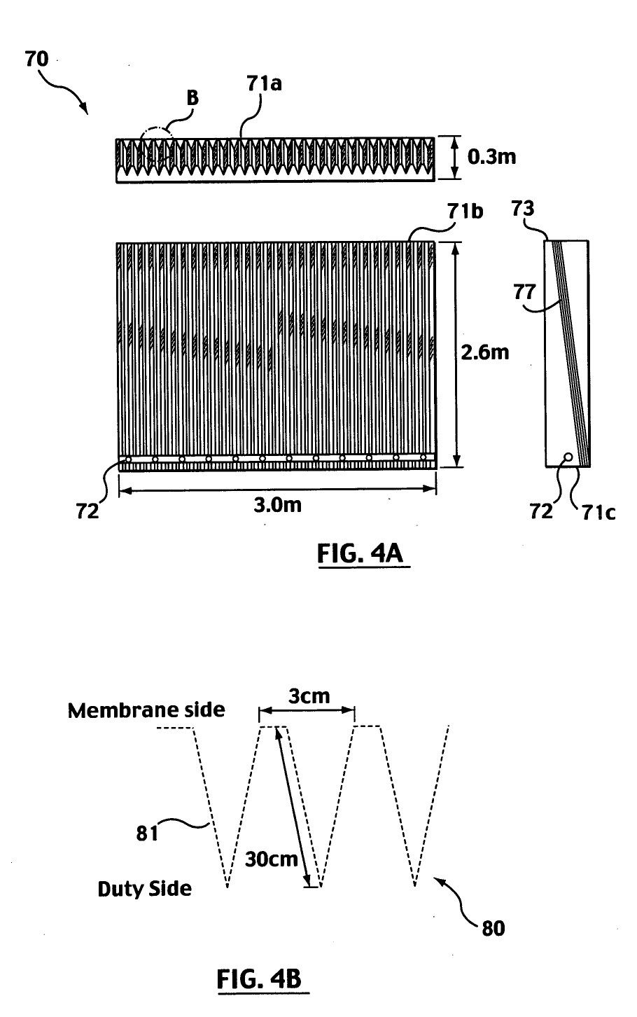

The static screen 35 includes a coarse bubble aerator 38 for gas scouring and gas flow induced backwashing. Aerator 38 is coupled to receive pressurized gas (typically from an air blower) through aeration stream 40. Details relating to two specific examples of static screens according to aspects of embodiments of the invention are provided further below with reference to FIGS. 3, 4A and 4B. Large screen 93 may be constructed like the embodiments of FIGS. 3, 4A and 4B but at an increased width.

The membrane tank 25 houses a number of membrane assemblies 37a, 37b, 37c and 37d that are placed downstream of the static screen 35 (i.e. in the second portion of the membrane tank 25). In some embodiments the membrane assemblies are in a cassette form, such as, for example, a ZW-500d cassette available from Zenon Environmental Inc. As shown in FIG. 2B, the membrane tank 25 may also be re-arranged, for example by providing a static screen 35 along one or both lengths of the membrane tank 25 to provide larger static screens 35 for membrane assemblies 37 of the same membrane surface area. Optionally, the static screens 35 may surround the membrane assembly 37 on all four sides in plan view with primary RAS 26 withdrawn through the floor of the membrane tank 25 below the membrane assemblies 37. Further optionally, the static screens 35 may encapsulate the membrane assemblies 37, for example by providing screening surfaces 35 or non-porous surfaces 35b on all 6 sides of a rectangular cassette of membrane assemblies 37, preferably with primary RAS 26 withdrawn by pipe passing through a static screen 35 and with screen backwashing by backwashing the membrane assembly 37.

The membrane tank 25 also includes two drains 51, 52. A larger primary drain 51 is located upstream of the static screen 35 and a smaller secondary drain 52 is located downstream of the static screen 35. The primary and secondary drains 51, 52 share a fluid connection to a drain valve 54, which is in fluid communication with a common sump 56. With further reference to FIGS. 1A, 1B, 1C and 1D, the common sump 56 (not shown in these Figures) may receive drainage from all or a plurality of the membrane tanks 21, 23 and 25. The common sump 56 is in fluid communication with a common drain pump 59. The common drain pump 59 is arranged to output a RAS/WAS (Waste Activated Sludge) stream from the collection of membrane tanks 21, 23 and 25 via the common sump 56.

In operation, mixed liquor enters the membrane tank 25 on the inlet side of the membrane tank 25 upstream of the static screen 35 (i.e. in the upstream section 110 of the membrane tank 25). The static screen 35 serves to filter out a substantial portion of roped and balled bundles of hair and the like from the mixed liquor entering the membrane tank 25 before the mixed liquor is permitted to flow to the membrane assemblies 37a, 37b, 37c and 37d. The roped and balled bundles of hair and the like that are caught by the static screen 35 and are flushed eventually through the fluid connection to the common secondary RAS stream 28, which may be designed, for example, to support a flow generally equal to average inlet flow rate Q of the waste water treatment system 10, for example between 0.5 and 1.5 Q. Moreover, periodic reverse flows to clean the static screen 35 may also take place employing the fluid connection to the secondary RAS stream 28, or direct mixing with the aerobic zone 16 in FIG. 1D, to return sludge flowing in a reverse direction through the screen to the bioreactor 14. The embodiment of FIG. 1C operates similarly but with adjustments for the location of the large screen 93.

The mixed liquor that flows through the static screen 35 or large screen 93 flows through the membrane assemblies 37a, 37b, 37c and 37d that are each made up of a number of membrane fibers. Consequently, the static screen 35 or large screen 93 protects the membrane assemblies 37a, 37b, 37c and 37d by continuously screening the mixed liquor directly before the mixed liquor is introduced to the membrane assemblies 37a, 37b, 37c and 37d. The membrane fibers are hollow and porous, which allows clarified water, known as permeate, from the mixed liquor to flow into the hollow interiors of the membrane fibers. The filtered permeate water is then drawn from the membrane tank 25 via a permeate stream into the effluent stream 24.

The aeration stream 40 is delivered to each of the membrane assemblies 37a, 37b, 37c and 37d. The aeration stream 40 is coupled to the bottom of each of the membrane assemblies 37a, 37b, 37c and 37d and releases bubbles to provide air scouring for the respective membrane fibers (not shown). The aeration stream 40 is also connected to coarse bubble aerators 38 below the static screens 35 to provide bubbles which contact and rise past the static screens 35. This helps reduce and delay fouling of the static screens 35 and to float retained solids to the secondary RAS stream 28. Alternately, separate aeration streams 40 may be provided to the membrane assemblies 37a, 37b, 37c, 37d and the static screen 35. Air, or other gases, in the one or more aeration streams 40 may be provided continuously, intermittently or cyclically. Air valves 41 may be operated to allow air, or other gases, to be provided to the screen 35 or membrane assemblies 37, or both, at any given time. For example, the supply of gases may be provided to the membrane assemblies 37 for most, for example between 50% and 95%, of operation time, and intermittently diverted to the screen 35. Alternately, gases may be supplied to the membrane assemblies 37 without regard to the needs of the screen 35, which is aerated when desired without regard to the needs of the membrane assemblies 37. However, since aerating the screen 35 reduces the density of water upstream of the screen 35, which interferes with flow of liquids to the membrane assemblies 37, the screen 35 may be aerated only periodically, for example directly before and/or during a screen 35 backwash as described below. Alternately, or additionally, the screen 35 may be aerated periodically with sufficient intensity to cause a backwash of the screen 35 by reducing the density of water upstream of the screen 35. Liquids backwashed through the screen 35 during intense aeration may flow to the secondary RAS channel 28 or mix with an upstream zone or other part of the total system. These comments, and others referring to one screen 35, apply to the other screens 31, 33, 93.

For example, a screen 35 in an embodiment as shown in FIG. 2A may be operated with a maximum head loss to flow through the screen of 15 to 30 cm. During normal operation of the screen 35, liquid flows through the screen 35. While liquid flows through the screen, air is provided to the aerator 38 of the screen 35 at a rate between about 0.5 and 2.0 scfm per horizontal linear foot of screen 35. This provides some cleaning of the screen 35 without causing an unacceptable head reduction for flow though the screen 35. During this time, very little, if any, liquid or solids overflows into the secondary RAS stream 28. Air may also be provided to the membrane assemblies 37 during this time as desired. Periodically, for example between about once a minute and once an hour, the screen 35 may be backwashed by providing a higher rate of aeration. For example, air may be provided to the aerator 38 of the screen 35 at a rate between about 8 and 12 scfm per horizontal linear foot of screen 35, for a backwash period of between about 5 to 20 seconds. If necessary, the air valves 41 may be operated to divert air from the membrane assemblies 37 to provide the increased airflow to the screen 35. This higher rate of aeration causes a decrease in the density of the liquid upstream of the screen 35, or otherwise causes liquid to flow backwards through the screen 35. Simultaneously, solids and liquid are floated or flow upwards upstream of the screen 35 and overflow into the secondary RAS stream 28. After the backwash period, the rate of aeration returns to the lower level to resume normal forward flow of liquid through the screen 35. The membrane assemblies 37 may be backwashed just before or while the increased airflow is provided to assist in backwashing the screen 35. In water filtration systems which typically have larger membrane surface areas in relation to the influent flow Q, of flow into screening apparatus 100, than wastewater plants, the volume of water added to the downstream section 112 during a membrane backwash may be significant and may even be sufficient to backwash the screen 35 alone.

Sludge that is not extracted through the membrane fibers from the membrane tank 25 generally flows through the fluid connection to the common primary RAS stream 26, although some is wasted through the drains 51, 52.

In an additional, optional, cleansing process, the static screen 35 (as well as static screens 31 and 33) can be purged by backwashing and draining solids from upstream of the static screens 31, 33, 35. In order to do this the drain valve 54 is opened and the mixed liquor flows out through the primary and secondary drains 51 and 52, respectively. Since the primary drain 51 is larger than the secondary drain 52 a larger amount of the mixed liquor flows through the primary drain 51 causing the mixed liquor in the membrane tank 25 to flow in the opposite direction through the static screen 35 than it normally flows when the drain valve 56 is closed. At this time flow of mixed liquor through exit line 22 may be slowed or stopped or the drain flow rates may be made to exceed the mixed liquor flow rate through exit line 22. Reversing the flow of the mixed liquor through the static screen 35 removes at least some of the trash, debris, grime, fibers, etc. that have collected on the upstream side of static screen 35. At least some of this released material, as well as solids too dense to be floated to secondary RAS stream 28, are drained out of the area upstream of the static screen 35. Alternatively, this operation can be facilitated by pumps that can be controlled to cause a reversal in the normal direction of a mixed liquor flow through one or more of the membrane tanks 25. The membrane assemblies may be backwashed directly before or during the draining to assist in backwashing the screen 35.

FIG. 3 shows an example of parts of a static screen 35, specifically a flat panel static screen 60. A top view of the flat panel static screen 60 is indicated by 61a, a front view of the flat panel screen is indicated by 61b and a side view is indicated at 61c. A flat solid plate, not shown, may be added to the top of the flat panel static screen 60 to provide a non-porous surface 356.

The flat panel static screen 60 is designed to be housed in a stainless steel frame having dimensions that fit snugly, and preferably with or allowing for a perimeter seal, to a membrane tank 21, 23, 25 which in turn fits closely to the membrane assemblies 37. The dimensions specified herein are provided for example only and relate to ZW-500d cassettes available from Zenon Environmental Inc. The thickness of the flat panel static screen 60, as seen in the top view 61a, is 0.3 m but may be larger, for example 0.5 m. The front view 61b as illustrated in FIG. 3 shows that the height and width of the screen are 2.6 m and 3.0 m, respectfully. Height may be increased, for example to 3.0 metres to allow for deeper water levels 108 if desired. Other sizes may be used as appropriate for other membrane assemblies 37, 38 and 39 which are employed in various facilities.

The flat panel static screen 60 consists of one or more flat panels having punched holes or a sheet of wire mesh 67 optionally positioned at an angle to the vertical with the top of the one or more panels leaning upstream. The holes in the flat panel(s) or wire mesh are sized to filter roped and balled bundles of hair or trash and the like in a waste water treatment system. In some embodiments the holes may be 0.5 mm to 1.0 mm in diameter or smallest width. In other embodiments the holes may be specified to be smaller than 3 mm. In other embodiments, the hole diameter or smallest width may be 1000 microns or less, 500 microns or less, 250 microns or less, 100 microns or less, or 50 microns or less.

In some embodiments the effective cross-sectional area of the flat panel static screen is about 90% of the available area of the front surface of the static screen. In the specific example illustrated in FIG. 3, the effective area is approximately 7.0 m² or 75 ft².

The flat panel static screen 60 also includes a coarse bubble aerator 62. The coarse bubble aerator 62 provides air scouring to reduce the build-up of trash, debris, grime, etc. on the one or more flat panels or wire mesh 67 during operation and to help float solids to the secondary RAS stream 28 or provide a backwash as described above.

Flat panel static screens are preferably used in facilities that have membrane tanks that are relatively wide in comparison to the membrane surface area or have low recycle rates, for example as FIG. 1B or 1E.

Provided as a second example of a static screen, shown in FIG. 4A is a schematic diagram illustrating various views of an undulating panel static screen 70. Specifically, a top view of the undulating panel static screen 70 is indicated by 71a, a front view of the undulating panel screen is indicated by 71b and a side view is indicated at 71c. Moreover, shown in FIG. 4B is a schematic diagram illustrating a detail view, generally indicated by 80, of a portion of the undulating panel static screen shown in FIG. 4A indicated by B in FIG. 4A.

Similar to the flat panel static screen 60, the undulating panel static screen 70 is designed to be housed in a stainless steel frame having dimensions to generally fill the vertical cross-section of the tank 21, 23, 25 and preferably to abut or be sealable to the insides of the walls of the tanks 21, 23, 25. The frame facilitates prefabrication of the static screen 60, 70, installation to or removal from anchor points in a tank, lifting for example by crane and integration of the aerator 62, 72.

The undulating panel static screen 70 consists of one or more flat panels having punched holes or a sheet of wire mesh 77 optionally positioned at an angle to the vertical, with the top of the one or more panels leaning upstream. As for the flat panel static screen 60, this angle helps the air bubbles scour the wire mesh 77 and also narrows the gap between the screening surface and back wall of the tank to increase water velocity during a backwash into the secondary RAS 28 channel. The holes may be of the same sized described for the flat panel static screen 60.

With further reference to FIG. 4B, the undulating panel static screen 70 is made up of a number of flat panels or panel sections 81 (each having holes as described above) arranged in an undulating or zigzag pattern. In the specific example illustrated in FIG. 4B each of the flat panels or panel sections 81 is 30 cm wide and the regular intervals between the flat panels is 3 cm. By arranging the flat panels 81 in this fashion a significantly larger effective surface area is provided by the undulating panel static screen 70 in comparison to the flat panel static screen 60 illustrated in FIG. 3. For example, the surface area may be 2, 5 or 10 times or more greater than the surface area of a flat panel. In the specific example illustrated in FIG. 4A, the effective surface area is approximately 88 m² or 950 ft². The undulating panel static screen 70 also includes a coarse bubble aerator 72 located at the bottom of the frame as described for the flat panel static screen 60.

Undulating panel static screens provide more effective area for screening mixed liquor for a given fixed cross-sectional area. Undulating panel static screens, or other configurations having a high SSAratio, are preferably used in facilities where membrane tanks 25 closely confine respective membrane assemblies 37 and are narrow compared to the membrane surface area or recycle rate, particularly as in FIGS. 1A, 1C and 1D. The undulating surface permits a higher flow-through rate for a given pressure drop and is fouled at a slower rate than a comparable flat panel static screen having the same cross-sectional area.

With reference to the example of static screens 60 and 70 illustrated schematically in FIG. 3, 4A and 4B, respectfully, in some embodiments the static screens 60 and 70 can be advantageously pre-fabricated. Thus, each static screen can be designed and installed as a package with an associated membrane assembly that has a cassette structure having a defined set of available dimensions, such as for example, the ZW-500d noted above. Moreover, static screens according to aspects of embodiments of the invention can be sized and pre-manufactured to be installed in existing waste water treatment systems with little or minimal changes to existing membrane tanks 25.

FIG. 2C shows another treatment system 95 having a bioreactor 14 and a membrane zone 12 and a screening apparatus 100 integrated into a membrane tank 25. The size of the downstream section 112 of the membrane tank 25 containing the membrane assemblies and the bioreactor have been reduced in the Figure to allow the upstream section 110 to be drawn larger. Flow from the bioreactor 14 to the membrane zone 12 is by pump 302 in the exit line 22. RAS 26, 28 flows by gravity back to the bioreactor 14 through pipes with check valves 304. The upstream and downstream sections 110, 112 of the combined membrane tank 25 and screening apparatus 100 are separated by a partition 300 that also acts as a non-porous surface 35b of a static screen 35. The static screen 35 has a set of open-topped mesh cylinders 306, having 0.75 mm openings, which function as screening surfaces 35a. The cylinders 306 are connected to a header 308 having an outlet 310 passing through the partition 300 to the downstream section 112. The top 10-20 cm of the cylinders 306 have non-porous sections 312. These non-porous sections inhibit bubbles, from aerators 38, trapped against the bottom of header 308 from being forced through the screening surface 35a before they flow around the header 308. Alternately, the structure may be inverted with the cylinders 306 extending upwards from the header 308 and the aerators 38 resting on the header 308. However, having the header 308 above the cylinders 306 allows for draining the upstream area 110 without draining the downstream area 112. In particular, when the downstream water level 108b goes below the bottom of the outlet 310, the upstream area 110 may be drained by opening drain 51 only without water flowing from the downstream section 112 through the static screen 35. The downstream water level 108b may be reduced to or below the bottom of outlet 310 by, for example, particularly with exit line 22 closed, draining the upstream section 110, permeating or draining from the downstream section 112, or recycle flow 26 from the downstream section 112. The upstream section 110 may be aerated to scour or shake material from the static screen 35 before draining. Such a process may be useful, for example as an alternate regular cleaning method, as a method used from time to time to remove solids from the upstream section 110 that cannot be floated over the overflow 118 or at the end of a night or low flow operation mode described earlier in which solids have been allowed to accumulate in the upstream section 110. Drained material may be, for example, further processed, wasted or recycled.

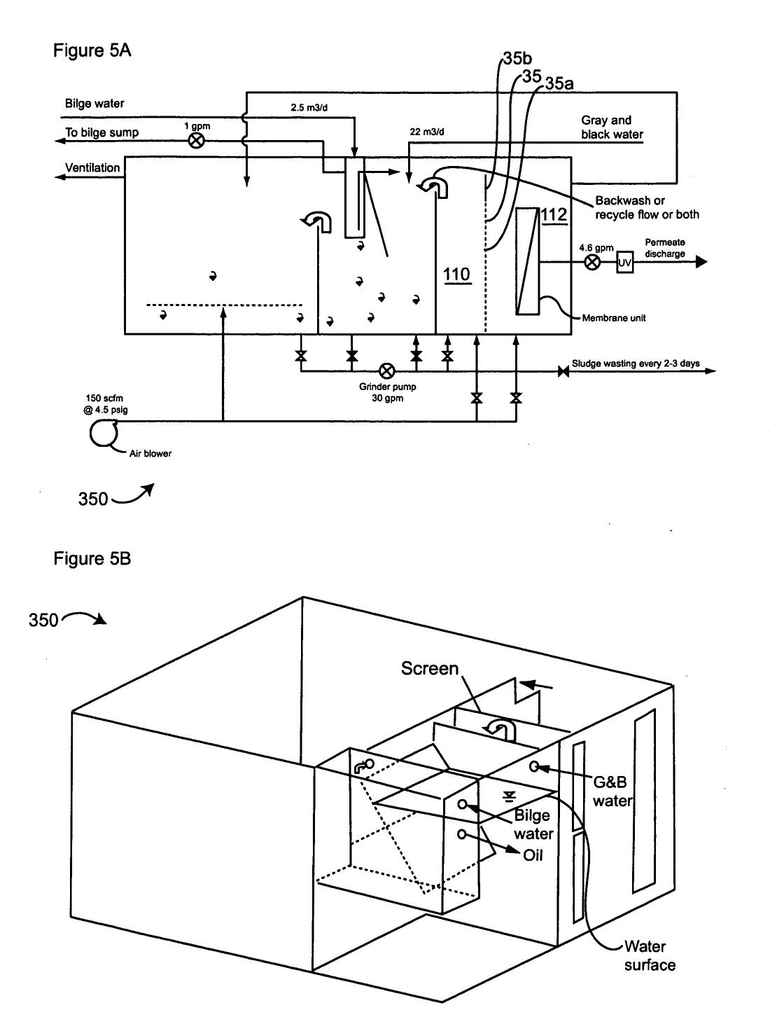

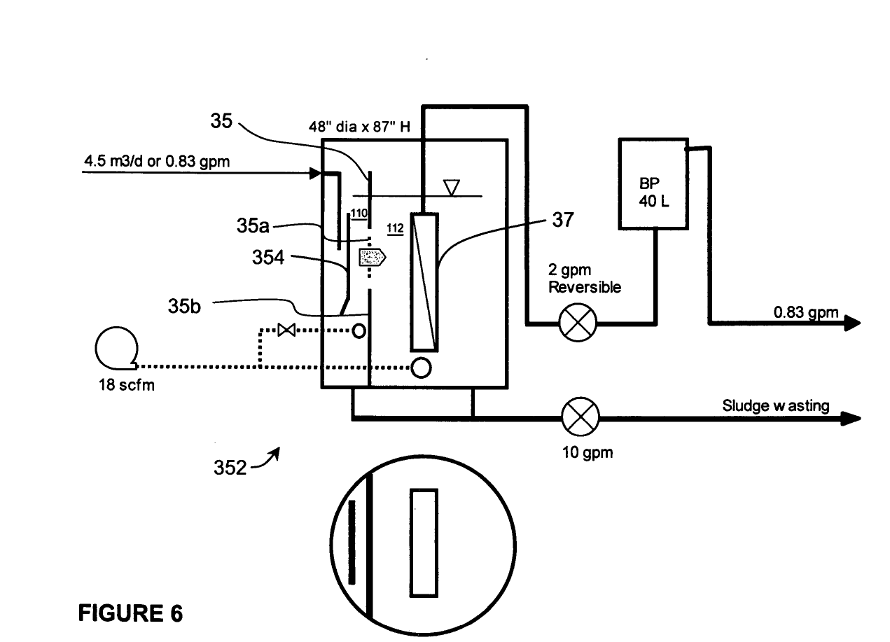

FIGS. 5A, 5B and 6 show shipboard MBR wastewater treatment systems, although their designs may also be useful for other small wastewater treatment plants. Shipboard MBR systems may treat grey and black water, including or excluding bilge water. These systems face serious challenges because of, for example, the following constraints: 1) the limited space available, for example with a maximum deck height of 7-8 ft; 2) an external screen is undesired for smell concern and solids handling; 3) pure oxygen is not preferred especially for naval ships and 4) there may be no sludge wasting for 2-45 days. System simplicity and compactness are important.

A shipboard waste water system 350 shown in FIGS. 5A and 5B aims to treat all the shipboard grey, black and bilge waters in one system. This system has the following features: 1) a coalescing step included to remove free oil from the bilge water; 2) a static screen according to any of the static screens described herein, and any of the air cleaning or backwashing processes described herein for a screen, to remove trashes and solids; 3) a mechanism implemented to collect and waste the trash, oil and grease, scum and foam; 4) a mechanism included to secure sufficient sludge left after sludge wasting to prevent the system from control failure or any wrongdoing and 5) vertical ZeeWeed™ membranes applied with cyclic air souring.

This design was originated for ships but the concept may be applicable to other small MBR plants, especially the ISO containerized.

The system 350 consists of a process tank, a sludge transfer pump, a free oil discharge pump, a blower, a permeate pump and a UV unit. The process tank is partitioned into a bilge water coalescing chamber, a trash and O&G collection chamber, a bioreactor chamber and a membrane chamber. The trash and O&G collection chamber is further divided into an upper part and a lower part by a slopped baffle and separated from the bioreactor chamber by a dividing weir that also divides the bioreactor volume into an upper portion and a lower portion.

The bilge water is pumped by a positive displacement pump from the bilge sump to the bilge water coalescing chamber where the large oil globules are separated by gravity in the first section of the chamber and the residual oil is separated in the second section where the coalescing materials assist the oil globules to join together and migrate/rise to the surface. The free oil is collected in the upper part of the chamber and discharged periodically back to the bilge sump by the free oil discharge pump upon the inlet bilge water flow rate while the decanted water flows into the trash and O&G collection chamber, blended with the incoming grey & black water. If the bilge water is not included to treat, the bilge water coalescing chamber can be removed and the system becomes a grey and black water treatment device.

In the trash and O&G collection chamber, the oil & grease stay in the upper part while the trashes and large solids settle down to the lower part of the chamber. The blended water passes through the underneath of the sloped baffle and enters into the bioreactor chamber over the dividing weir. Due to the low velocity across the weir and the sufficient height of the weir, most settled trashes and solids will not be carried over to the bioreactor chamber such that the large solids content in the bioreactor chamber is minimized which protects the static screen and its aerators.

The aerobic bioreactor chamber is aerated by a means of medium bubble aerators in order to compromise the oxygen transfer rate with foaming potential. Because of the short water depth, the aerobic chamber is sized to ensure the oxygen transfer rate for carbonaceous BOD/COD removal. Antifoam may be added to control foaming, if necessary.

The sludge transfer pump transfers the mixed liquor from the bioreactor chamber to the static screen channel in the membrane chamber where the mixed liquor penetrates the static screen and enters into the membrane zone while the solids rejected by the screen are carried back to the trash and O&G collection chamber during the screen backwash period. The oxygen-enriched sludge with the trash from the screen channel prevents the trash and O&G collection chamber from becoming anaerobic and also performs organic biodegradation. Supplemental air may be added periodically or continuously to the trash and O&G collection chamber for gentle mixing and oxygen supply, if necessary.

In the membrane zone, the clean water is drawn out through the membranes and the excess trash-free sludge overflows back to the bioreactor chamber. Vertical membrane modules may be applied with cyclic air scouring. In case of limited tank height, the membrane modules will be shortened to the available water depth.

The accumulated sludge is wasted on a regular basis as required, directly from the trash and oil and grease (O&G) collection chamber. The collected trash and oil and grease, and the scum and foam, if any, in the bioreactor chamber are discharged so that the trash/solids accumulation and the foaming potential are minimized. The sludge transfer pump is specified as grinder pump for trash handling and continuously chops the solids carried to the bioreactor chamber and discharges trash when wasting sludge. Prior to sludge wasting, the sludge transfer pump is operated in a closed loop within the trash and O&G collection chamber to mix the settled trashes and solids and then this chamber is completely emptied to discharge the collected trashes, oil and grease with sludge wasting. In the mean time, the scum and foam in the aerobic chamber, if any, flow through the dividing weir back to the trash and O&G collection chamber and are also wasted with the sludge.

The weir height between the bioreactor chamber and O & G collection chamber is determined based upon the sludge holding time and the design mixed liquor concentration such that the total volume of the trash and O&G chamber plus the upper portion of the bioreactor volume above the dividing weir is equal to the sludge volume to be wasted and sufficient sludge is kept in the bioreactor chamber for system operation after sludge wasting.

An air blower is included to provide air for the operations of ZeeWeed™ membranes, the static screen and the bioreactor chamber. A UV unit may further disinfect the discharged effluent.

The static screen is included in the membrane chamber to remove the solids carried with sludge and bring the solids back to the trash and O&G collection chamber during the backwash period so that the membrane sludging is reduced. The excess trash-free sludge from the membrane zone overflows back to the bioreactor chamber. The static screen actually serves as a side screen to transfer the trashes from the bioreactor to the trash and O&G collection chamber. Water, O&G and trash separation performances in the trash and O&G collection chamber is improved because of reduced hydraulic load and solid concentration.

In one example for use in a naval ship, the grey & black and bilge water treatment on board ship is as shown in FIGS. 5A and 5B. The system is to treat 22 m³/d grey and black water and 2.5 m³/d bilge water within a limited space. There is no sludge wasting for 2-3 days. The combined influent is assumed to have a BOD5 of 960 mg/L and TSS of 900 mg/L.

The proposed system is designed to fit a 20′ ISO container. The process tank is sized as 13.5° L×7′ W×7.1° H, with an overall volume of 18.9 m³. At a water depth of 1.72 m in the aerobic chamber, the total aerobic volume is 13.2 m³, which gives a HRT of 12.9 hrs. The process tank is partitioned as:

TABLE 2

| | Bioreactor | Membrane | Trash & O&G | Bilge |
|---|---|---|---|---|
| Chamber | Chamber | Chamber | Chamber | water |
| Length, m | 2.87 | 1.25 | 1.25 | 1.25 |
| Width, m | 2.13 | 0.75 | 1.38 | 0.25 |
| Water depth, m | 1.72 | 1.87 | 1.72 | 0.97 |
| Liquid volume, m³ | 10.5 | 1.75 | 2.25 | 0.30 |

The dividing weir height is set to 1.4 m, giving the upper portion of the aerobic chamber a volume of 1.93 m³. The volume of this portion plus the volume of the trash and O&G chamber is about 29% of the total liquid volume. At a MLSS concentration of 14 g/L before sludge wasting, this partition results in a MLSS concentration of 10 g/L after sludge wasting. The system has a SRT of 5.5-10 days and is able to hold sludge for 3 days.

Due to the limited water depth of 1.87 m in the membrane chamber, the standard ZW-500™ modules are not applicable. Therefore, ZW-500d™ modules by Zenon Environmental Inc. are shortened to fit the ZW chamber and the ZW chamber itself serves as the support frame. With six (6) such modified ZW-500d™ modules, an average permeate flux of about 4-5 gfd is assumed to provide a conservative design suitable for use under difficult conditions.

For most small systems, especially ISO containerized systems, standard membrane modules are too tall to install. Without extending the membrane tank above the container, the modules should fit a water depth of less than about 1.8 m (or 6′). Shortened ZeeWeed™ modules or other modules of this size may be side mounted directly to the membrane chamber walls.

FIG. 6 shows another small shipboard gray and black water treatment system 352. The static screen 35 is largely oversized being 2 ft by 2 ft with a design capacity of 0.25 gpm/ft². Accordingly, the static screen operates under minimal head differential. The screen 35 otherwise operates as described generally above except that aerating the upstream section 110 causes an air lift on the screen 35 side of partition 354 that may cause a backwash but also circulates water around the partition 354. The membrane unit 37 may optionally be backwashed or relaxed at the same time as the screen 35 is backwashed. The membrane assembly 37 is kept soaking in sludge and maintenance cleaned once a day.

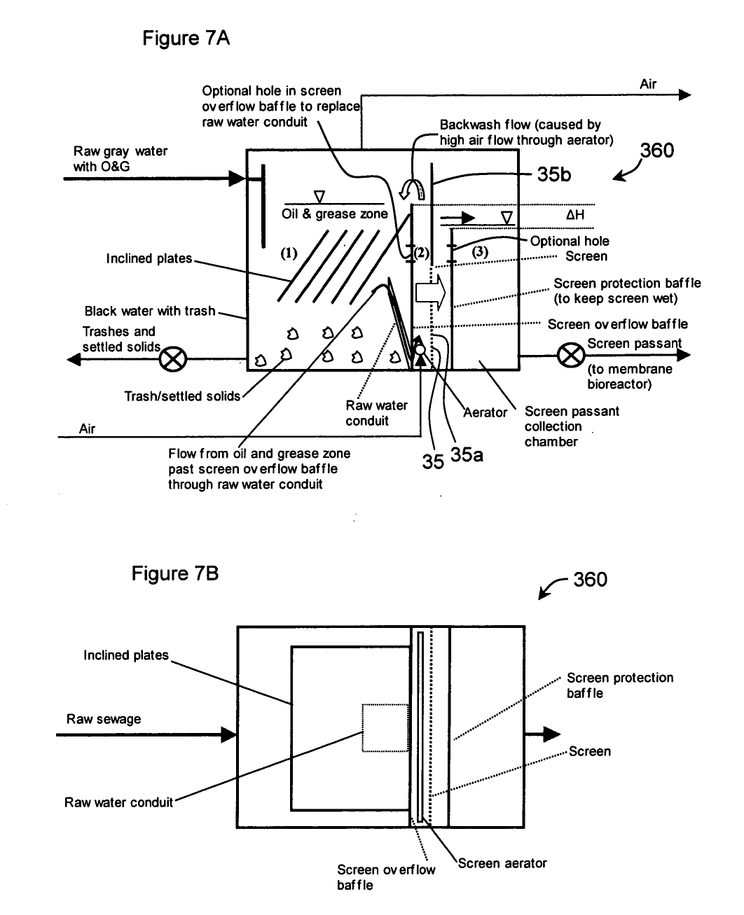

Another small system is shown in FIGS. 7A and 7B which may be called a primary screen-clarifier 360. The primary screen-clarifier 360 uses a static screen 35 according to any of the embodiments described previously and air cleaning or backwashing processes described herein for a screen to replace the external primary screening equipment for small shipboard or other small MBR or other applications. This principle may be applicable to other small MBR systems. The advantages of this application include; substantial removal of trashes and BOD/TSS from the raw sewage, low hydraulic load to the screen (1 Q instead of 4-5 Q when the recirculating mixed liquor is screened), low solid load to the screen (influent TSS instead of MLSS), no screening solids to handle since the settled solids are pumpable, no smell concern because the clarifier tank can be fully closed and ventilated, and compact and low cost. Tests indicated that a screen has higher loading capacity with raw sewage than with mixed liquor allowing a small screen size in combination with the low hydraulic load.

The primary screen-clarifier 360 consists of a clarifier chamber 1, a static screen chamber 2 and a screen passant collection chamber 3. The screen chamber 2 is located between the clarifier chamber 1 and the screen passant collection chamber 3 and formed with a screen overflow baffle and a static screen. A bundle of inclined plates are installed in the clarifier chamber 1 to minimize hydraulic turbulence and lead the settled solids to the bottom of tank. In the mean time, a screen protection baffle is installed in the screen passant collection chamber 3 to ensure the screen 35 is always submerged and provide the sufficient water for screen 35 backwash. Backwash may be by flow of air to the aerator sufficient to cause a temporary reverse flow through the screen. The optional hole in the screen overflow baffle in place of the raw water conduit helps, if used, draw water from the clean side of screen 35 rather than O&G zone during backwash.

Once raw wastewater enters into the clarifier chamber 1, the trash and large particulates settle down to the bottom of the tank while oil & grease float to the top. Medium size solids may be suspended and carried over with the water stream to the screen chamber 2 through a water conduit. The water conduit is installed behind the inclined plates at a reasonable height to avoid taking any oil & grease to the screen chamber 2. The water stream penetrates the static screen 35 while the suspended solids are rejected and returned during the backwash period back to the clarifier chamber 1 where the solids settle onto the inclined plates and drop to the bottom of the tank. Screen passant may flow to a membrane bioreactor or other downstream treatment stage.

The trashes and the solids accumulated in the clarifier chamber 1 are discharged periodically to maintain a reasonable solid concentration in the clarifier chamber 1. The entire clarifier chamber 1 will be fully emptied once a while to dispose of the accumulated oil & grease on the top of the chamber.

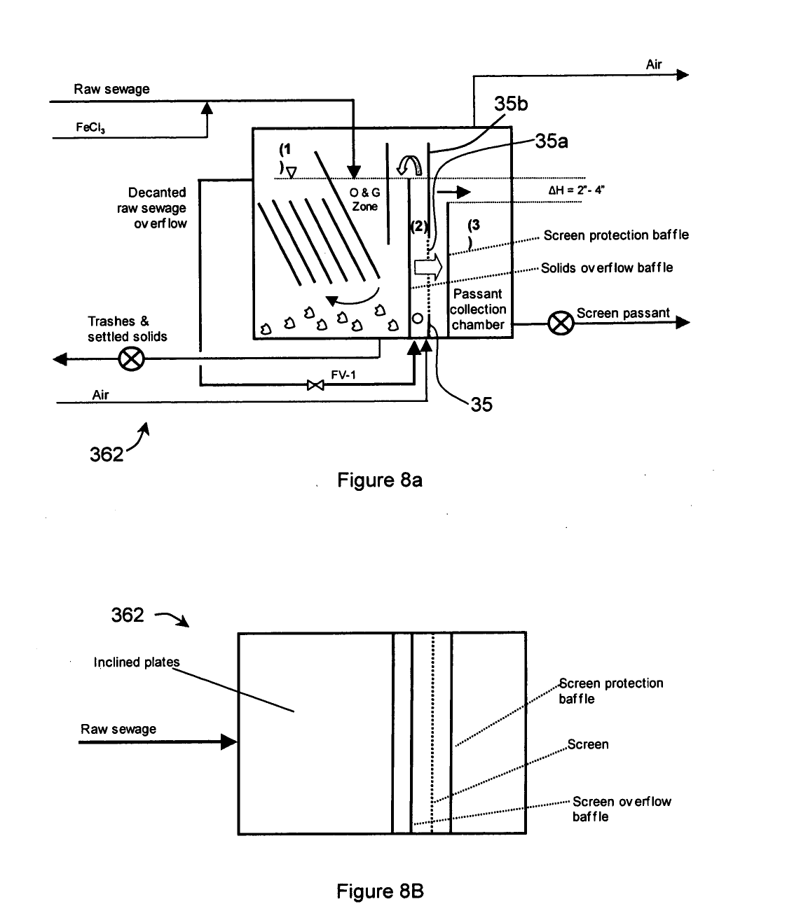

As shown in FIGS. 8A-8B, a second primary screen clarifier 362 consists of a clarifier chamber 1, a static screen chamber 2 and a passant collection chamber 3. The screen chamber 2 is located between the clarifier chamber 1 and the passant collection chamber 3, formed with a solids overflow baffle and a static screen. A separate baffle in the clarifier chamber forms an O&G zone with inclined plates and also prevents air-scouring turbulence. A screen protection baffle in the passant collection chamber ensures the screen 35 is always submerged and that there is sufficient water for backwash.

When the raw sewage enters into the O&G zone of the clarifier chamber 1, the trash and large solids settle down to the clarifier bottom while oil & grease remain on the top. The water flows down with the rejected solids from the screen chamber 2 and then up through the inclined plates (70°) where the solids may settle but the water continues to flow to the overflow port and then to the screen chamber 2. The water penetrates through the static screen 35 while the suspended solids are rejected and returned back to the clarifier chamber during a backwash period caused by aeration as described above. An optional automatic valve (FV-1) on the sewage overflow pipeline can be closed during the backwash period, if necessary.

The trash or solids accumulated at the clarifier bottom and the oil & grease on the top of the chamber are discharged periodically.

This configuration minimizes the possible hydraulic turbulence in the O&G zone and the inclined plate zone so that the water stream entering into the screen chamber is well decanted and trash free, with minimal free O&G. The returned solids stream from the screen chamber during the backwash period also has reduced turbulence impact on the clarifier chamber 1.

A sample application is a ZeeWeed MBR system for a ship that generates about 40 m³/d grey and black water with an average TSS of 1000 mg/L. The MBR system must hold solids and sludge (no discharge) for 45 days within a limited space. For this reason, the bioreactor will have to allow the mixed liquor concentration built up from 5 g/L to 35 g/L during this period.

An issue with a long solids/sludge holding time is trash accumulation, which at the high MLSS makes it difficult to have a static screen in the membrane chamber. Therefore, a primary screen clarifier is applied to remove the trash and solids and reduce the BOD/TSS load to the bioreactor. Ferric chloride is also added to improve solids settling. The primary screen clarifier is 2 m W×1.3 m D×2.0 m H, with total volume of 5.2 m³. Assuming 20% TSS removal and 2.5% settled trash/solids (DS) at the clarifier bottom, the clarifier will discharge 320 L/d solids to a trash holding tank (14.4 m³ for 45 days).

All the internal baffles can be bolted instead of welded to facilitate the tank fabrication because watertight baffles are not necessary. The primary screen clarifier for the above application is partitioned as below:
Clarifier chamber 1: 1.22 m W×1.3 m D×2 m H
   Liquid volume: 3.33 m³ @ 1.7 m H
Passant collection chamber 3: 0.58 m W×1.3 m D×2 m H
   Liquid volume: 0.64 m³ @ 1.65 m H
Screen chamber 2: 0.075 m W×1.3 m D×2 m H
   Screen size: 1.3 m×1.4 m H
   Screen surface area: 1.82 m²

The main process parameters for this application are as follows:
Clarifier Surface Overflow Rate (SOR): 25.3 m³/m²/d
Clarifier Solids Loading Rate (SLR): 25.3 kg/m²/d
Clarifier HRT: 2 hr
Screen hydraulic loading: 0.47 gpm/ft² @ 80% effective area
Inclined plate angle: 60-70°
Trash/solids discharge: twice a day
O&G discharge: once every two weeks

If the second primary screen clarifier 362 is applied to a small air transfer membrane system as shown in, for example, International Publication No. WO 2004/071973, which is incorporated herein in its entirety, the screen protection baffle can be removed and the gas transfer modules can be installed in the passant collection chamber because of the low TSS concentration.

A number of design examples for various systems are presented below in Examples 1 to 6.

TABLE 3
MBR Mixed Liquor—Small System (FIG. 1E)
MBR Mixed Liquor—Small System

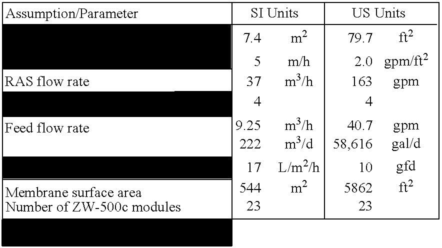

Example 1 is reflected in Table 3 which applies to a system as in FIG. 1E. It was assumed for Example 1 that a flat screen is mounted directly into a tank and that mixed liquor is recirculated by pumping from the membrane compartment. In this design, a low loading rate (2 gpm/ft²) is used as it is assumed that trash accumulates in the mixed liquor (i.e. no headworks screen) and is removed with excess sludge. This design is to limited systems having approximately one single cassette of ZW-500 membrane modules.

TABLE 4
MBR Mixed Liquor—Large System (FIG. 1A)
MBR Mixed Liquor—Large System

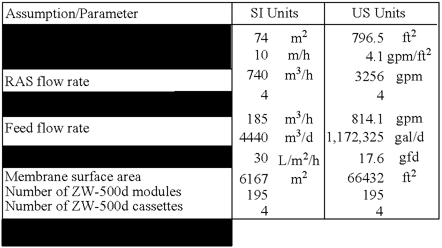

Example 2 is described in Table 4 and relates to a system as in FIG. 1A. A large surface area screen is assumed and mixed liquor is pumped to the membrane tank. Fine headworks screening requirements, typically 6 mm, can be relaxed or even eliminated if a side-stream screen is used to extract trash from the aeration tank. With a demonstrated loading rate of 4 gpm/ft², the static screen could handle up to a 4 cassette membrane tank.

TABLE 5
Primary Wastewater Screen-Clarifier (Figures 8A—8B)
Primary Wastewater Screen-Clarifier

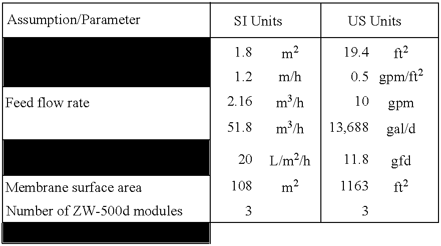

Example 3 is described in Table 5 and relates to a system as in FIGS. 8A and 8B. This design is proposed for a screen built into a clarifier for pretreatment in a shipboard system. This is an alternative to the design presented in Example 1 for small systems.

TABLE 6
Surface Water—Membrane Protection (FIG. 1B)
Surface Water—Membrane Protection

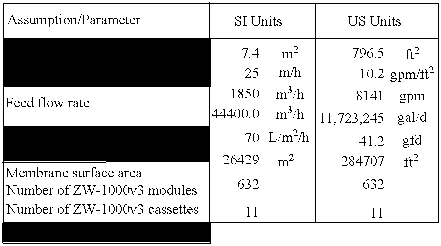

Example 4 is described in Table 6 and relates to a system as in FIG. 1B. A 100 mm screen and relatively high loading rate of 10 gpm/ft² were assumed in this design example. In a preliminary test, described below, this screen did not remove turbidity or coagulated solids from surface water but will protect the membranes. With a SSAratio=10 screen, very large ZW-1000v3 applications could be handled.

TABLE 7
Surface Water—Algae/Floc Removal (FIG. 1B)
Surface Water—Algae/Floc Removal

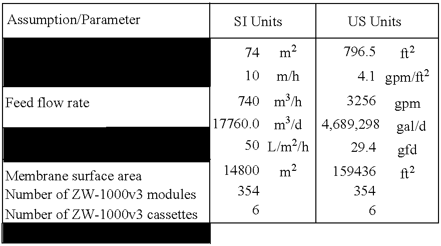

Example 5 is described in Table 7 and relates to a system as in FIG. 1B. To remove algae and floc from surface water, a 38 mm screen and loading of 4 gpm/ft² were assumed. This application appears feasible for ZW-1000v3 tanks with up to 3 cassettes.

Examples 1 to 5 are summarized in Table 8 below.

TABLE 8
Summary system design examples 1-5

| | Screen | | | |
|---|---|---|---|---|
| | Characteristics | | | |
| | | | Loading rate | |
| | Size | | m/h | Plant Size |
| Application | (μm) | SSAᵣₐₜᵢₒ | (gpm/ft²) | (screen at the front of tank) |
| 1 - MBR mixed liquor | 750 | 1.0 (flat) | 5.0 (2.0) | Max 222 m³/d (60,000 gal/d) |
| Small system | | | | with 4Q ML recycling |
| | | | | 1 (one) ZW-500c cassette |
| 2 - MBR mixed liquor | 750 | 10.0 | 10.0 (4.1) | Max tank 4440 m³/d (1.1 MGD) |
| Large system | | | | with 4Q ML recycling |
| | | | | 4 ZW-500d cassettes |
| 3 - Raw wastewater | 750 | 1.0 (flat) | 1.2 (0.5) | Up to 100 m³/d (26,400 gal/d) |
| Screen - Clarifier | | | | On settled wastewater |
| 4 - Surface water | 100 | 10.0 | 25 (10.2) | Max tank 45,000 m³/d (12 |
| Membrane protection | | | | MGD) |
| | | | | 11 ZW-1000v3 cassettes with |
| | | | | flux of 70 L/m²/h (40 gdf) |
| 5 - Surface water | 38 | 10.0 | 10.0 (4.1) | Max tank 18,000 m³/d (5 MGD) |
| Algae/floc removal | | | | 6 ZW-1000v3 cassettes with |
| | | | | flux of 50 L/m²/h (30 gdf) |

Example 6 describes a design for backwashing a static screen by draining a tank having a screening apparatus integrated with a membrane tank, generally as shown in FIG. 2A used in a system as shown in FIG. 1A designed as a 12,000 m³/d plant.

The membrane tank contains 5 ZW-500d cassettes (flux of 20.5 μm²/h). Assuming a length of 3 m (10 ft) per cassette, the membrane tank has a cross section of 46.5 m² (500 ft²) and a volume of 140 m³ (37,000 gal).

Total ML flow to each tank is ⅓ of 5 Q, with 4 Q through the screen and 1 Q overflow to the secondary RAS channel:
ML flow to a membrane tank: 20,000 m³/d (5.28 MGD)
ML flow through the screen: 16,000 m³/d (4.22 MGD)

A corrugated screen is specified having a surface area of 88 m² or 950 ft² and a loading rate of 7.5 m/L or 3.1 gpm/ft².

The static screen is air scoured during forward screening. Trash is carried to the secondary RAS overflow channel with the ML cross flow of 1 Q.

Additional trash may be discharged through the secondary RAS channel by closing a gate from the membrane area to the primary RAS channel during relaxation or backwash of the membranes.

The volume of ML upstream of the screen is about 3.0 m³ (800 gal), corresponding to a 13 cm (5 inches) drop of membrane tank water level when the screen is backwashed (to displace 2 volumes of the section upstream of the screen).

The screen can be backwashed by doing a partial tank drain and concentrated trash discharged to a sump tank to feed a side-stream screen. Use can be made of a tank drain pump, which is designed to empty the tank in 30 min (280 m³/h (1230 gpm))

For a backwash, the level in the upstream section of the tank may be made to drop below that of the downstream section in one of two ways:
(1) Stop or reduce significantly the flow of ML to the membrane tank with a faster acting valve or stopping a feed supply pump. A backwash flow of 2 Q=360 m³/h (1584 gpm) could be handled by the tank drain pump
(2) Provide a large primary drain and a sump to remove the entire ML flow, plus the backwash flow. For a total flow of 7 Q (5Q ML+2 Q backwash), and a backwash duration of 1 min, the sump volume would be 20 m³ (5000 gal)

In Example 7, testing was done with a pilot unit equipped with a flat screen of 5.4 ft2 (0.5 m2) installed in a converted ZW-500d tank, without membranes installed, with a feed of mixed liquor. Screens of 0.5, 0.75 and 11.0 mm were evaluated. The 1.0 and 0.75 mm screen were found to remove substantially all trash and hair (about 80 mg/L). The 0.5 mm screen removed an additional 50 mg/L of paper fibres.

Loading rates of 4.0-5.3 gpm/ft² (10-13 m/h) were obtained with heads of 6 to 10 inches (15-30 cm). Sustainable operation could be obtained with a dead-end filtration time of 4 min and a 10 s backwash. The screen remained clean over several months of intermittent operation, with no mechanical/chemical cleaning.

In Example 8, raw sewage testing was done over a few weeks to validate the static screen for small shipboard systems. Loadings of 3-5 gpm/ft² were obtained for trash contents of 2-12 g/L. Oil and grease were added to simulate kitchen wastewater. The application was validated, but some regular screen maintenance would be required when the ship come to port and the tank is emptied.

In Example 9, the hydraulic permeability (tested with clean water) of 2 wire mesh fine screens was measured as showed in Table 9.

Both screens were tested on coagulated/flocculated surface water (45 mg/L PACI). The 100 mm screen removed no or insignificant amount of solids and the measured loading rate was close to that of clean water. The 38 mm screen removed about 50% of the TSS with loading rates of 3-5 gpm/ft² under up to 6 inches of head.

TABLE 9
Clean water permeability of fine screens

| Screen mesh | Hydraulic Permeability |
|---|---|
| opening (μm) | (gpm/ft²/inch of water head) |
| 38 | 4.3 |
| 100 | 20.7 |

In Example 10, to investigate the process functionality of static screening of Mixed Liquor (ML), a study was conducted at a waste water treatment plant. Using a static screen pilot, screen size openings of 0.5-mm, 0.75-mm and 1-mm, having a surface area of 5.4 ft² (0.5 m²) were tested. Various conclusions from this testing are stated below.

Static screen is a technically viable option for in-tank screening of mixed liquor. A simple backwash method, based on increasing air flow upstream of the screen, works well. Aeration energy requirement is very low, and long term trash accumulation on the surface of the screen appears manageable.

For a ML with a given trash concentration, the net loading rate depends on continuous air flow rate upstream of the static screen (dirty side), differential head at backwash and screen size opening. The achievable net loading rates under optimum operating conditions are:

| | |
|---|---|
| 0.5-mm screen | 4 gpm/ft² (9.8 m/h), |
| 0.75-mm screen | 5 gpm/ft² (12.3 m/h) |
| 1-mm screen | 5.3 gpm/ft² (13 m/h) |

Three differential heads at backwash (end of filtration cycle), 6.5″, 8″ and 10″ were tested. There exists a relationship between screen size opening and optimum differential head at backwash. Finer screen size openings have higher optimum differential head at backwash. For 0.5-mm, 0.75-mm and 1-mm screen sizes, the optimum differential heads at backwashes are 10″, 8″ and 6.5″, respectively.

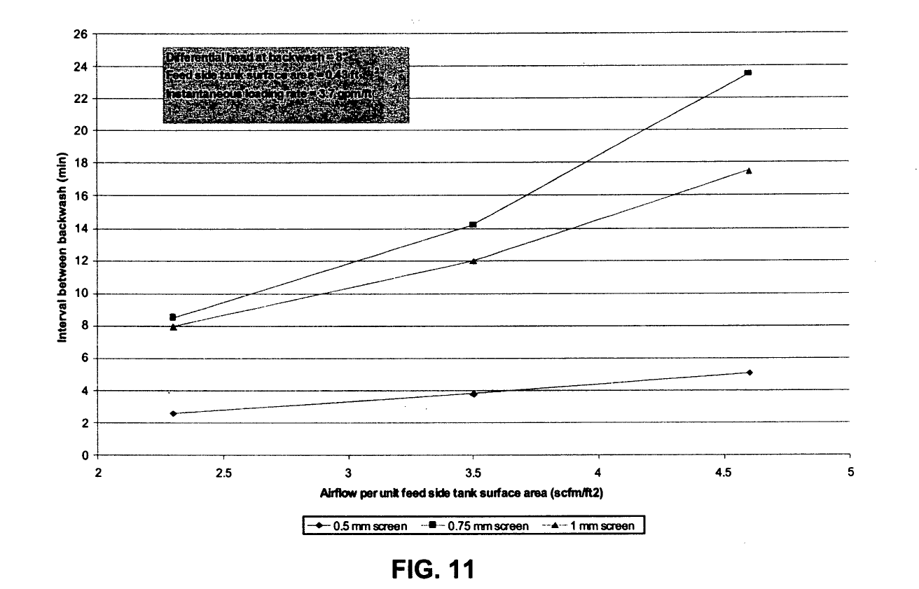

Increasing the continuous airflow per unit feed side tank surface area (0.1 m×0.4 m) increases the filtration cycle time (increases the interval between backwash). FIG. 11 shows the impact of increased airflow or filtration (screening) cycle time at a differential head at backwash of 8″ and an instantaneous loading rate of 3.7 gpm/ft² (9.1 m/h).

For the screen size of 1-mm at a differential head at backwash of 6.5″, increasing the backwash airflow per unit feed side tank surface area from 23.2 to 30.2 scfm/ft², didn't increase the net loading rate significantly.

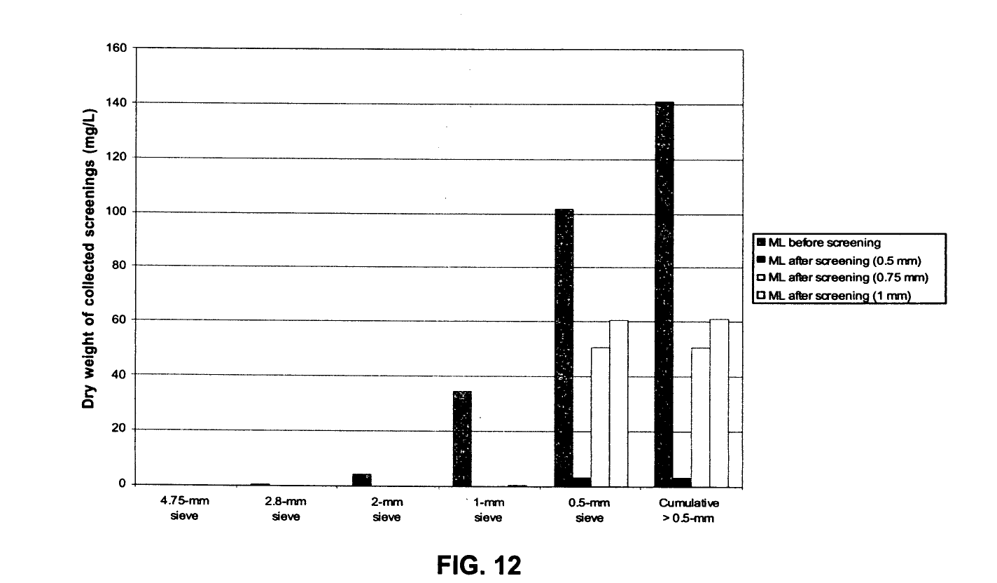

Mixed liquor tested has a trash content of approximately 140 mg of screenings per L of mixed liquor. FIG. 12 shows that 0.5-mm, 0.75-mm and 1 mm static screens removed all material 1-mm and larger from the mixed liquor, as expected, and also that the 0.5-mm screen removed more material in the range of 0.5-1.0 mm than the 0.75-mm and 1 mm screen did. Results show that 0.75-mm and 1-mm screens are capable of removing material smaller than their effective screen size opening.

Based on knowledge gained from the pilot study, the following recommendations are made for large mixed liquor screening systems such as in FIG. 1A:
Screen size opening: 0.75 mm
Differential head at backwash: 6.5″
Net loading rate: 4.2 gpm/ft²(10.3 m/h)
Filtration (forward screening) cycle time: 4 minutes
Backwash duration: 10 seconds
Continuous airflow per unit feed side tank surface area: 3.5 scfm/ft² (63.7 Nm³/m²/h)
Backwash airflow per unit feed side tank surface area: 23.2 scfm/ft² (422.2 Nm³/m²/h)
Feed side width (upstream of screen): 2″ to 5″

The last three parameters are based on 4.3″ (0.1 m) tank width and flat screen configuration tested in a pilot. These parameters may change for other screen configuration in a full-scale application.

In Example 11, a study conducted on a static screen pilot with a screen size opening of 38 μm, and a surface area of 5.5 ft², provided observations or conclusions as described below.

A 38 μm static screen with a clean water permeability of about 170,000 gfd/psi is a viable option for solids removal in ZW 1000 applications. Solids removal ranged between 32% and 52% for feed water with TSS ranging from 45-47 mg/L. The backwash sequence, based on increasing air flow upstream of the screen, works very well. Aeration energy requirement is very low, and accumulation on the surface of the screen during a filtration cycle was negligible.

For flocculation water with a TSS ranging from 45-47 mg/L and a differential head at backwash of 6.5″, the highest achievable loading rate depends on continuous air flow rate upstream of the static screen. Under optimum operating conditions (i.e. constant air flow=2-2.5 scfm), the highest achievable loading rate for cycle times>10 minutes is 3.2-3.46 gpm/ft².

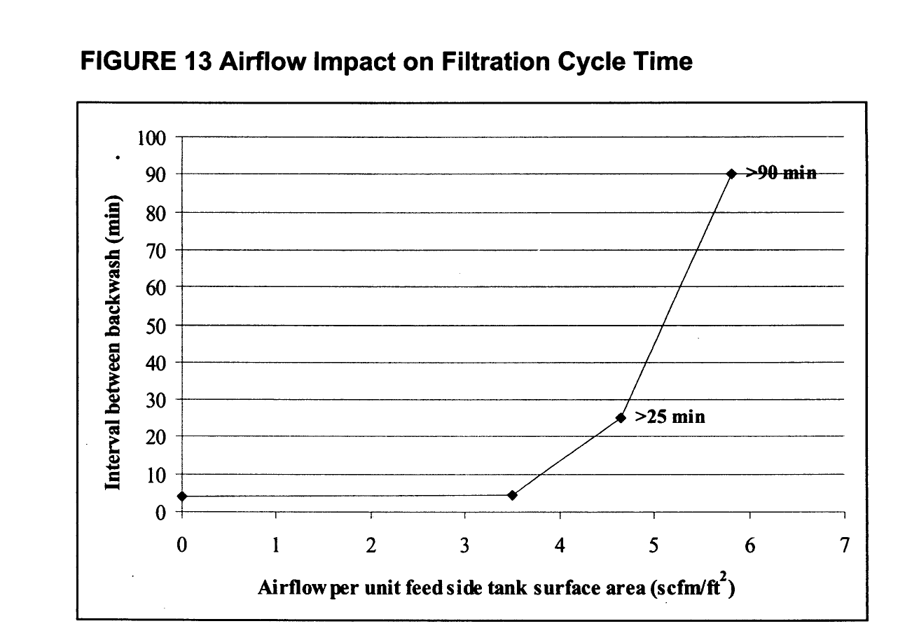

Increasing the continuous airflow per unit feed side tank surface area (0.33 ft×1.31 ft) increases the interval between backwash. FIG. 13 shows the impact of increased airflow at a differential head at backwash of 6.5″ and an instantaneous loading rate of 3.2 gpm/ft². For a loading rate of 3.27 gpm/ft², increasing the continuous airflow per unit feed side tank surface area up to 5.81 scfm/ft² allows for filtration cycles>90 min.

For the screen size tested (38 μm) and a differential head at backwash of 6.5″, increasing the backwash airflow duration from 10 to 20 seconds significantly increased the filtration cycle time for a given loading rate. For example, at a loading rate of 2.67 gpm/ft², increasing the backwash airflow duration from 10 to 20 seconds increased the cycle time from 3 to greater than 10 minutes.

The results of these tests are presented in Tables 10 and 11:

TABLE 10
Test 1
Feeding Flocculated Water (TSS = 45 mg/L)
38 μm screen (5.35 ft²)
BW sequence (15 scfm/10 s)

| | | | | | TSS |
|---|---|---|---|---|---|
| | Flow Rate | Constant Air | Cycle Time | H* | Removal |
| Test | (gpm/ft²) | (2 scfm) | (minutes) | infl./effl. | (%) |
| 1 | 3.2 | N | 0 | 32/33 | — |
| | 3.2 | N | 1 | 30/33 | 51 |
| | 3.2 | N | 3 | 28/33 | — |
| | 3.2 | N | 3.75 | OF | — |
| 2 | 3.2 | N | 0 | 32/33 | — |
| | 3.2 | N | 1 | 30/33 | — |
| | 3.2 | N | 3 | 27.75/33 | — |
| | 3.2 | N | 4 | OF | — |
| 3 | 3.2 | Y | 1 | 30/33 | 32 |
| | 3.2 | Y | 3 | 30/33 | 45 |
| | 3.2 | Y | 25 | 30/33 | 48 |
| 4 | 6.1 | Y | 0.5 | OF | — |
| 5 | 4.7 | Y | 0 | 30/33 | — |
| | 4.7 | Y | 1 | 30/33 | — |
| | 4.7 | Y | 2 | 30/33 | — |
| | 4.7 | Y | 4 | 29/33 | 50 |
| | 4.7 | Y | 5 | OF | — |
| 6 | 4.7 | Y | 0 | 30/33 | — |
| | 4.7 | Y | 1 | 30/33 | — |
| | 4.7 | Y | 2 | 30/33 | — |
| | 4.7 | Y | 3 | 29.5/33 | — |
| | 4.7 | Y | 4 | 29/33 | — |
| | 4.7 | Y | 5 | OF | — |

*Distance from the top of the tank to the water surface
Note:
overflow located 27.5″ from the top of the tank
backwash conducted between tests

TABLE 11
Test 2
Feeding Flocculated Water (TSS = 47.3 mg/L)
38 μm screen (5.35 ft²)

| | | Constant | Cycle | B.W. | TSS |
|---|---|---|---|---|---|
| | Flow Rate | Air | Time | Condition | Removal |
| Run | (gpm/ft²) | (scfm) | (minutes) | scfm/sec. | (%) |
| 1 | 1.87 | 1.5 | >12 | 15/10 | 18 |
| | | | | (1″)* | (t = 10 min) |
| 2 | 2.67 | 1.5 | 3 | 15/10 | — |
| 3 | 2.67 | 1.5 | >10 | 15/20 | 34 |
| | | | | (4-5″)* | (t = 4 min) |
| 4 | 3.27-3.46 | 1.5 | 4.02 | 15/20 | — |
| 4 (repeat) | 3.27 | 1.5 | 4.67 | 15/20 | 40 |
| | | | | (5″)* | (t = 4 min) |
| 5 | 3.27 | 2.5 | >13 | 15/20 | 48 |
| | | | | | (t = 6 min) |
| 6 | 4.39 | 2.5 | 2 | 15/20 | — |
| 7 | 3.27 | 2.5 | >90 | 15/20 | 52 |
| | | | | | (t = 40 min) |

*water lost during backwash

In Example 12, to investigate the process functionality of static screening of raw sewage, tests were conducted using a static screen pilot with a screen size opening of 0.75-mm and having a surface area of 5.4 ft² (0.5 m²). Raw sewage was used as a feed source, with manual addition of trash (screenings retained on 0.5 mm sieve), oil and grease. The main conclusions of this study are summarized below.

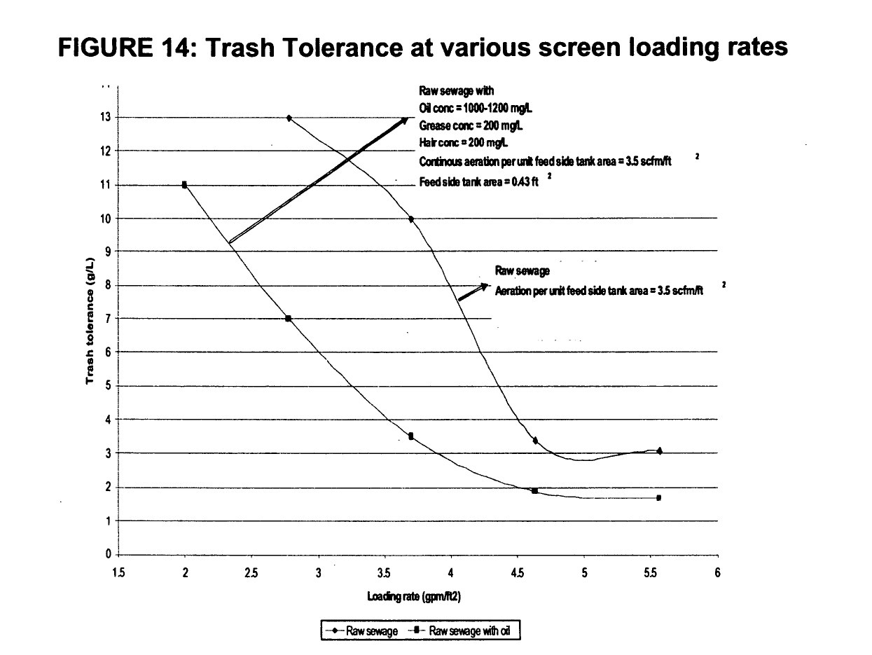

A static screen is a technically viable option for screening of raw sewage. Filtration rates are better than expected and trash tolerance of the system is also high at a given loading rate. FIG. 14 is generated for design purposes and to assess the process capacity. Each point of the curve in FIG. 14 represents the maximum trash concentration the system could handle at a given hydraulic loading rate, without an overflow from the dirty side of the screen i.e. all the flow is passing through the screen. The system had a trash tolerance of about 13 g/l at a screen loading rate of 2.78 gpm/ft², with a differential head at backwash of 6.5″. Continuous aeration of 3.5 scfm/ft² per unit feed side tank area was provided to keep the screen clean and to keep the trash in suspension.

At a given screen loading rate, the trash tolerance was reduced when the process fluid was raw sewage with oil (1000-1200 mg/l) and grease (200 mg/l). FIG. 14 shows the curve generated under these conditions and shows the comparison between raw sewage alone and raw sewage with oil and grease, as a process fluid (the operating conditions in terms of continuous aeration and differential head at backwash were the same). However, even with difficult process water (oil and grease), the performance of static screen was quite good and the system had a trash tolerance of about 11 g/l at a screen loading rate of 2 gpm/ft² (FIG. 1).

The pilot was operated continuously for about 12 days, having oil concentration of 1000 mg/l and grease concentration of 200 mg/l. The screen loading rate was 3.5 gpm/ft² and feed trash concentration was 350 mg/l. Filtration cycle time was 5 minutes and backwash was for 10 seconds. There were no serious operational issues and a backwash method based on increasing air flow upstream of the screen worked well. However, after 4 or 5 days of operation very fine balls of grease started forming, though there was no compromise on system performance. The screen was inspected after 12 days of continuous operation. A very fine matting (less than 1 mm) was observed at the top ½ of the screen. However, the matting was observed to be quite permeable and was easily removed by water hose.

A 0.75 mm screen retains almost 100% of hair. The pilot was operated with hair concentration 200 mg/l in the feed and no hair was found downstream of the screen.

FIG. 14 can be used for design purposes. At an appropriate design screen loading rates and knowing the approximate trash concentration in the feed, filtration cycle time or backwash frequency can be estimated.

What has been described above is merely illustrative of embodiments of the invention. Other arrangements of elements or steps can be implemented by those skilled in the art, without departing from the scope of the invention, which is defined by the following claims.

We claim:

1. A screening apparatus for use in a water treatment system having an upstream area under ambient pressure with a first static head and a downstream area under ambient pressure with a second static head, the screening apparatus comprising:
one or more generally static screening surfaces having a plurality of openings, wherein any dimension of the openings is approximately 3 mm or less;
a structure for holding the screening surface in communication with the upstream and downstream areas such that the screening surface intercepts water flowing between the upstream and downstream areas; and,
one or more aerators in communication with the upstream area.

2. The screening apparatus of claim 1 wherein the downstream area is a membrane tank.

3. A screening apparatus according to claim 1 further comprising an outlet for retained screenings from the upstream area.

4. A screening apparatus according to claim 1 wherein the smallest dimension of the openings is 1 mm or less, 100 μm or less or 50 μm or less.

5. A screening apparatus according to claim 1 wherein the one or more screening surfaces are in the shape of open ended three-dimensional bodies.

6. A screening apparatus according to claim 1 having a non-porous section extending at least from below a water surface level of the downstream section to above a water surface level of the upstream section.

7. A screening apparatus according to claim 1 wherein the upstream area has a volume that is 30% or less of the downstream volume.

8. A screening apparatus according to claim 1 wherein the area of the one or more screening surfaces exceeds the area of the largest vertical cross-section of the screening apparatus by a factor of 2 or more.

9. A screening apparatus according to claim 1 wherein the one or more screening surfaces communicate with one or both of the upstream and downstream areas through a conduit, plenum, header or manifold.

10. A screening apparatus according to claim 1 having an overflow from the upstream area to a waste or recycle stream.

11. A screening apparatus according to claim 1 further comprising a drain from the upstream area.

12. A screening apparatus according to claim 1 having a gas supply connected to one or more aerators, the gas supply configured to provide a gas at a rate that varies between a first rate and a second rate, the second rate being in the range between no flow and about one half of the first rate.

13. An apparatus having a screening apparatus according to claim 1 and further comprising:
a tank having an inlet; and,
a membrane assembly immersed in the tank,
wherein the screening apparatus is located so as to intercept water flowing to the inlet or from the inlet to the membrane assembly.

14. An apparatus comprising:
one or more fluidly connected tanks;
an inlet to the one or more tanks;
a membrane assembly immersed in one of the tanks;
a static screen separating a volume of water containing the membrane assembly from the inlet;
a permeate outlet connected to the membrane assembly; and,
a membrane retentate outlet in communication with the volume of water containing the membrane assembly.

15. The apparatus of claim 14 further comprising an outlet from the tank containing the static screen from outside of the volume containing the membrane assembly.

16. The apparatus of claim 14 wherein the static screen is in direct communication with the volume of water containing the membrane assembly.

17. A water treatment system having an apparatus according to claim 14 and a water treatment area upstream of the screening surface.

18. A water treatment system according to claim 17 wherein the water treatment area is a biological treatment area.

19. A water treatment system according to claim 17 having a recycle between an upstream side of the screening surface and the water treatment area.

20. A water treatment system according to claim 17 comprising an oil and grease floatation zone or a settling zone upstream of the static screen.

21. A process for treating water comprising the steps of:
a) flowing water containing undesirable solids, the undesirable solids being at least 20 μm wide in any direction, through a generally stationary screening surface in a forward direction from an upstream side of the screening surface to a downstream side of the screening surface, the flow of the water driven substantially by the difference between a static head in communication with the upstream side of the screening surface and a lesser static head in communication with the downstream side of the screening surface; and,
b) stopping the flow of water through the screen in the forward direction from time to time and removing undesirable solids from the upstream side of the screening surface while the flow of water through the screen in the forward direction is stopped.

22. A process according to claim 21 wherein step (b) further comprises a step of backwashing the screening surface.

23. A process according to claim 21 further comprising steps of providing an upstream area through which water flows to the upstream side of the screen and removing between about 0.25 and 2.5 times the volume of the upstream area from the upstream area during step (b).

24. A process according to claim 21 further comprising a step of aerating the water on the upstream side of the screening surface.

25. A process according to claim 24 wherein the step of aerating is performed during step (b).

26. A process according to claim 24 wherein the step of aerating is performed at a rate that varies between a first rate used during step (b) and a second rate used during step (a), wherein the second rate is in the range of no flow to one half of the first rate.

27. A process according to claim 24 further comprising a step of flowing water from the downstream sides of the screening surface to a membrane assembly.

28. A process according to claim 21 further comprising a step of relaxing or backwashing the membrane assembly during step (b).

29. A process according to claim 21 further comprising a step of flowing water from downstream of the membrane assembly to upstream of the screening surface.

30. A process according to claim 21 further comprising a step of withdrawing water containing solids rejected by the membrane assembly from the downstream side of the screening surface.

31. A process according to claim 21 further comprising a step of recycling water containing undesirable solids removed in step (b) to a point further upstream of the screening surface.
